(19) United States
(12) Patent Application Publication
(10) Pub. No.: US 2024/0341726 A1
(43) Pub. Date: Oct. 17, 2024

(54) SOFT AND STRETCHABLE COMPOSITE MATERIAL WITH TUNABLE ACOUSTIC IMPEDANCE

(71) Applicant: NUtech Ventures, Lincoln, NE (US)

(72) Inventors: Eric Markvicka, Lincoln, NE (US); Sequoia Truong, Lincoln, NE (US); Kiersten Reeser, Lincoln, NE (US); Ethan Krings, Lincoln, NE (US); Greg Bashford, Lincoln, NE (US); Benjamin Hage, Lincoln, NE (US); Megan Santamore, Lincoln, NE (US); Caden Pak, Lincoln, NE (US)

(21) Appl. No.: 18/638,126

(22) Filed: Apr. 17, 2024

Related U.S. Application Data
(60) Provisional application No. 63/459,942, filed on Apr. 17, 2023.

(51) Int. Cl. A61B 8/00; A61B 8/08

(52) U.S. Cl. CPC A61B 8/4236; A61B 8/0891

(57) ABSTRACT

A wearable ultrasound patch may include one or more layers, including a backing layer, a transducer layer, a matching layer, and an adhesive layer. The matching layer may include liquid-metal microdroplets dispersed within an elastomer. A volume loading and/or diameter of the liquid-metal microdroplets may tune the acoustic impedance of the matching layer such that the matching layer may provide acoustic matching via a geometric mean between an acoustic impedance of piezoelectric elements of the transducer layer and a skin on which the wearable ultrasound patch is worn. The wearable ultrasound patch may be used in a system with a pulser-receiver.

### CROSS-REFERENCE TO RELATED APPLICATIONS

The present application claims the benefit under 35 U.S.C. § 119 (e) of U.S. Provisional Application Ser. No. 63/459,942, filed Apr. 17, 2023, titled “SOFT AND STRETCHABLE COMPOSITE MATERIAL WITH TUNABLE ACOUSTIC IMPEDANCE”, which is incorporated herein by reference in the entirety.

### STATEMENT REGARDING

This invention was made with government support under Grant No. 2050587 awarded by the U.S. National Science Foundation, Grant No. 2054411 awarded by the U.S. National Science Foundation, Grant No. 1542182 awarded by the U.S. National Science Foundation, and Grant No. 2054411 awarded by the U.S. National Science Foundation. The government has certain rights in the invention.

### TECHNICAL FIELD

The present disclosure generally relates to diagnosis using ultrasound waves, and more particularly to ultrasonic probe attachment by adhesive patches.

### BACKGROUND

Ultrasound is a safe, noninvasive diagnostic technique used to measure internal structures such as blood vessels and blood flow velocity in the human body. A mismatch of acoustic impedance at an interface between a piezoelectric transducer and a medium to be probed by the ultrasound may substantially reduce the amount of ultrasound energy being transmitted into the medium, and thus the intensity of the returned signal. The reduction in the intensity of the returned signal may cause low signal-to-noise.

Acoustic matching layers may improve the transfer of ultrasound energy from the transducer to the imaged medium. The matching layers may be rigid matching layers made of metal particle-polymer composites. These rigid matching layers may not be compatible with flexible and/or stretchable wearable ultrasound patches. Therefore, it would be advantageous to provide a device, system, and method that cures the shortcomings described above.

### SUMMARY

A wearable ultrasound patch is described, in accordance with one or more embodiments of the present disclosure. The wearable ultrasound patch may include: a transducer layer including an electrical circuit and a first elastomer, the electrical circuit including a ground plane, a plurality of feed lines, and a plurality of piezoelectric elements, wherein the ground plane includes a plurality of ground electrodes and a plurality of ground traces, wherein the plurality of feed lines include a plurality of feed electrodes, a plurality of feed traces, and an interface, wherein the plurality of piezoelectric elements couple the plurality of feed electrodes and the plurality of ground electrodes, wherein the plurality of piezoelectric elements are configured to generate a plurality of emitted ultrasound waves in response to receiving a plurality of input signals and generate a plurality of output signals in response to receiving a plurality of received ultrasound waves; and a matching layer including a second elastomer and a plurality of liquid-metal microdroplets, wherein the plurality of liquid-metal microdroplets are dispersed within the second elastomer.

In some aspects, the wearable ultrasound patch may include a backing layer, wherein the transducer layer is disposed between the backing layer and the matching layer.

In some aspects, the backing layer is abutted to the transducer layer.

In some aspects, the wearable ultrasound patch may include an adhesive layer, wherein the matching layer is disposed between the transducer layer and the adhesive layer.

In some aspects, at least one of: the matching layer is abutted to the transducer layer; or the adhesive layer is abutted to the matching layer.

In some aspects, the first elastomer embeds the ground plane, the plurality of piezoelectric elements, the plurality of feed electrodes, and the plurality of feed traces.

In some aspects, the transducer layer and the matching layer are configured to conform to a planar surface and a curved surface.

In some aspects, the matching layer includes an elastic modulus of between 10 kPa and 100 MPa.

In some aspects, the matching layer includes the elastic modulus of between 10 kPa and 1 MPa.

In some aspects, the plurality of liquid-metal microdroplets include a diameter between 10 nanometers and 500 micrometers.

In some aspects, the plurality of liquid-metal microdroplets include the diameter between 100 nanometers and 100 micrometers.

In some aspects, the matching layer includes an acoustic impedance between 1 and 7.2 MRayls.

In some aspects, the matching layer includes the acoustic impedance between 4.8 and 7.2 MRayls.

In some aspects, the matching layer includes the acoustic impedance between 5.6 and 5.8 MRayls.

In some aspects, the plurality of liquid-metal microdroplets include a volume loading between 10% and 80%.

In some aspects, the plurality of liquid-metal microdroplets include the volume loading between 50% and 70%.

In some aspects, the plurality of liquid-metal microdroplets include a eutectic gallium-based liquid metal.

In some aspects, at least one of the first elastomer and the second elastomer includes at least one of Polydimethylsiloxane, polyurethane, polyvinylsiloxane, butyl rubber, fluorosilicone, or styrene butadiene.

In some aspects, the eutectic gallium-based liquid metal includes one of eutectic gallium-indium (EGaIn) or a eutectic alloy of gallium, indium, and tin.

In some aspects, the plurality of liquid-metal microdroplets include a core and a liquid-metal shell, wherein the liquid-metal shell includes an alloy of a first metal and the eutectic gallium-based liquid metal.

In some aspects, the core includes at least one of tungsten, tungsten carbide, aluminum, copper, iron, nickel, or silver.

In some aspects, the first metal includes at least one of silver, copper, nickel, gold, or tin.

In some aspects, the plurality of liquid-metal microdroplets include an adhesive shell, wherein the adhesive shell is disposed between the core and the liquid-metal shell.

In some aspects, the adhesive shell includes polydopamine (PDA).

In some aspects the eutectic gallium-based liquid metal includes eutectic gallium-indium (EGaIn), wherein the core is a tungsten (W) core, wherein the first metal is silver (Ag), wherein the plurality of liquid-metal microdroplets are W@PDA-(Ag+EGaIn) microdroplets.

In some aspects, the wearable ultrasound patch is configured to perform acoustic beamforming of the plurality of piezoelectric elements by receiving the plurality of input signals at separate phases.

In some aspects, the plurality of piezoelectric elements are arranged in an array, wherein the array is a planar array.

In some aspects, the array is a five-row array with one of the plurality of piezoelectric elements in a first row, five of the plurality of piezoelectric elements in a second row, six of the plurality of piezoelectric elements in a third row, five of the plurality of piezoelectric elements in a fourth row, and two of the plurality of piezoelectric elements in a fifth row.

In some aspects, the matching layer includes an upper elastomer layer, a liquid-metal layer, and a lower elastomer layer, wherein the liquid-metal layer includes the second elastomer and the plurality of liquid-metal microdroplets, wherein the liquid-metal layer is disposed between the upper elastomer layer and the lower elastomer layer.

In some aspects, the liquid-metal layer is arranged in a lattice.

In some aspects, the upper elastomer layer abuts the transducer layer, wherein upper elastomer layer is thicker than the lower elastomer layer.

A system is described, in accordance with one or more embodiments of the present disclosure. The system may include a wearable ultrasound patch including: a transducer layer including an electrical circuit and a first elastomer, the electrical circuit including a ground plane, a plurality of feed lines, and a plurality of piezoelectric elements, wherein the ground plane includes a plurality of ground electrodes and a plurality of ground traces, wherein the plurality of feed lines include a plurality of feed electrodes, a plurality of feed traces, and an interface, wherein the plurality of piezoelectric elements couple the plurality of feed electrodes and the plurality of ground electrodes, wherein the plurality of piezoelectric elements are configured to generate a plurality of emitted ultrasound waves in response to receiving a plurality of input signals and generate a plurality of output signals in response to receiving a plurality of received ultrasound waves; and a matching layer including a second elastomer and a plurality of liquid-metal microdroplets, wherein the plurality of liquid-metal microdroplets are dispersed within the second elastomer; and a pulser-receiver, wherein the interface is connected to the pulser-receiver, wherein the pulser-receiver is configured to transmit the plurality of input signals to the interface and receive the plurality of output signals from the interface.

In some aspects, the pulser-receiver is configured to cause the wearable ultrasound patch to obtain one or more measurements of a mean velocity (\tilde{v}_{MCA}) of blood flow in a middle cerebral artery.

In some aspects, the system includes an additional wearable ultrasound patch; wherein the pulser-receiver is configured to cause the additional wearable ultrasound patch to obtain one or more measurements of a mean velocity (\tilde{v}_{ICA}) of blood flow in an internal carotid artery.

In some aspects, the additional wearable ultrasound patch comprises an additional transducer layer, wherein the additional transducer layer comprises an additional elastomer and an additional electrical circuit, wherein the additional electrical circuit comprises a top electrode, a bottom electrode, and a piezoelectric element.

A method is described, in accordance with one or more embodiments of the present disclosure. The method may include: spin-processing a matching layer, the matching layer including an elastomer and a plurality of liquid-metal microdroplets, wherein the plurality of liquid-metal microdroplets are dispersed within the elastomer; laser patterning a ground plane and a plurality of feed lines from one or more copper clad sheets, wherein the ground plane includes a plurality of ground electrodes and a plurality of ground traces, wherein the plurality of feed lines include a plurality of feed electrodes, a plurality of feed traces, and an interface; transferring the ground plane to the matching layer and the plurality of feed lines to a backing layer; forming a conductive epoxy on the plurality of ground electrodes and on the plurality of feed electrodes; placing a plurality of piezoelectric transducers between the plurality of ground electrodes and the plurality of feed electrodes; curing the conductive epoxy to bond the plurality of piezoelectric transducers between the plurality of ground electrodes and the plurality of feed electrodes thereby forming an electrical circuit of a transducer layer; and forming an elastomer of the transducer layer.

### BRIEF DESCRIPTION OF THE DRAWINGS

The numerous advantages of the disclosure may be better understood by those skilled in the art by reference to the accompanying figures in which:

FIG. 1A illustrates a perspective view of a wearable ultrasound patch, in accordance with one or more embodiments of the present disclosure.

FIG. 1B illustrates a top view of the wearable ultrasound patch, in accordance with one or more embodiments of the present disclosure.

FIG. 1C illustrates a side view of the wearable ultrasound patch, in accordance with one or more embodiments of the present disclosure.

FIG. 1D illustrates the side view of the wearable ultrasound patch emitting ultrasound waves in response to receiving input signals, in accordance with one or more embodiments of the present disclosure.

FIG. 1E illustrates the side view of the wearable ultrasound patch generating output signals in response to receiving ultrasound waves, in accordance with one or more embodiments of the present disclosure.

FIG. 1F illustrates a perspective view of an electrical circuit of the wearable ultrasound patch, in accordance with one or more embodiments of the present disclosure.

FIG. 1G illustrates an exploded view of the electrical circuit of the wearable ultrasound patch, in accordance with one or more embodiments of the present disclosure.

FIG. 2 illustrates a system including the wearable ultrasound patch, in accordance with one or more embodiments of the present disclosure.

FIG. 3 illustrates a flow diagram of a method for making the wearable ultrasound patch, in accordance with one or more embodiments of the present disclosure.

FIG. 4A illustrates a matching layer after spin-processing, in accordance with one or more embodiments of the present disclosure.

FIG. 4B illustrates a ground plane during removal of excess copper of copper clad sheets, in accordance with one or more embodiments of the present disclosure.

FIG. 4C illustrates the ground plane after being transferred onto the matching layer, in accordance with one or more embodiments of the present disclosure.

FIGS. 4D-4E illustrates coating a conductive epoxy onto ground electrodes of the ground plane using a first stencil mask, in accordance with one or more embodiments of the present disclosure.

FIG. 4F illustrates placing piezoelectric elements onto the ground electrodes of the ground plane before the piezoelectric elements are sandwiched between the ground plane and feed lines, in accordance with one or more embodiments of the present disclosure.

FIG. 4G illustrates the wearable ultrasound patch, in accordance with one or more embodiments of the present disclosure.

FIG. 5 illustrates a flow diagram of a method for spin-processing the matching layer, in accordance with one or more embodiments of the present disclosure.

FIG. 6 illustrates a cross-section view of the liquid-metal microdroplets, in accordance with one or more embodiments of the present disclosure.

FIG. 7 illustrates a flow diagram of a method of introducing tungsten into the liquid-metal microdroplets, in accordance with one or more embodiments of the present disclosure.

FIG. 8A illustrates a perspective view of the matching layer, in accordance with one or more embodiments of the present disclosure.

FIG. 8B illustrates an exploded view of the matching layer, in accordance with one or more embodiments of the present disclosure.

FIG. 8C illustrates a top view of the matching layer, in accordance with one or more embodiments of the present disclosure.

FIG. 9 illustrates a flow diagram of a method of manufacturing the matching layer, in accordance with one or more embodiments of the present disclosure.

FIG. 10A illustrates a perspective view of an additional wearable ultrasound patch, in accordance with one or more embodiments of the present disclosure.

FIG. 10B illustrates a top view of the additional wearable ultrasound patch, in accordance with one or more embodiments of the present disclosure.

FIG. 10C illustrates a side view of the additional wearable ultrasound patch, in accordance with one or more embodiments of the present disclosure.

FIG. 11 illustrates the system with the wearable ultrasound patch and the additional wearable ultrasound patch, in accordance with one or more embodiments of the present disclosure.

### DETAILED DESCRIPTION OF THE INVENTION

The present disclosure has been particularly shown and described with respect to certain embodiments and specific features thereof. The embodiments set forth herein are taken to be illustrative rather than limiting. It should be readily apparent to those of ordinary skill in the art that various changes and modifications in form and detail may be made without departing from the spirit and scope of the disclosure. Reference will now be made in detail to the subject matter disclosed, which is illustrated in the accompanying drawings.

Embodiments of the present disclosure are directed to a wearable ultrasound patch. The wearable ultrasound patch may include one or more layers, including a backing layer, a transducer layer, a matching layer, and an adhesive layer. The matching layer may include liquid-metal microdroplets dispersed within an elastomer. A volume loading and/or diameter of the liquid-metal microdroplets may tune the acoustic impedance of the matching layer such that the matching layer may provide acoustic matching via a geometric mean between an acoustic impedance of piezoelectric elements of the transducer layer and a skin on which the wearable ultrasound patch is worn. The wearable ultrasound patch may be used in a system with a pulser-receiver.

U.S. Pat. Pub. No. US20160176128A1, titled “Extremely Compliant Yet Tough Hydrogel Systems as Ultrasound Transmission Agents”; U.S. Pat. Pub. No. US20220175340A1, titled “System and method for continuous non-invasive ultrasonic monitoring of blood vessels and central organs”; U.S. Pat. Pub. No. US20220133269A1, titled “Integrated wearable ultrasonic phased arrays for monitoring; U.S. Pat. No. 11,116,448B1, titled “Multi-sensor wearable patch”; U.S. Pat. Pub. No. US20020161301A1, titled “Matching layer having gradient in impedance for ultrasound tranducers”; U.S. Pat. Pub. No. US20220363865A1, titled “Lightweight liquid metal embedded elastomer composite”; are each incorporated herein by reference in the entirety.

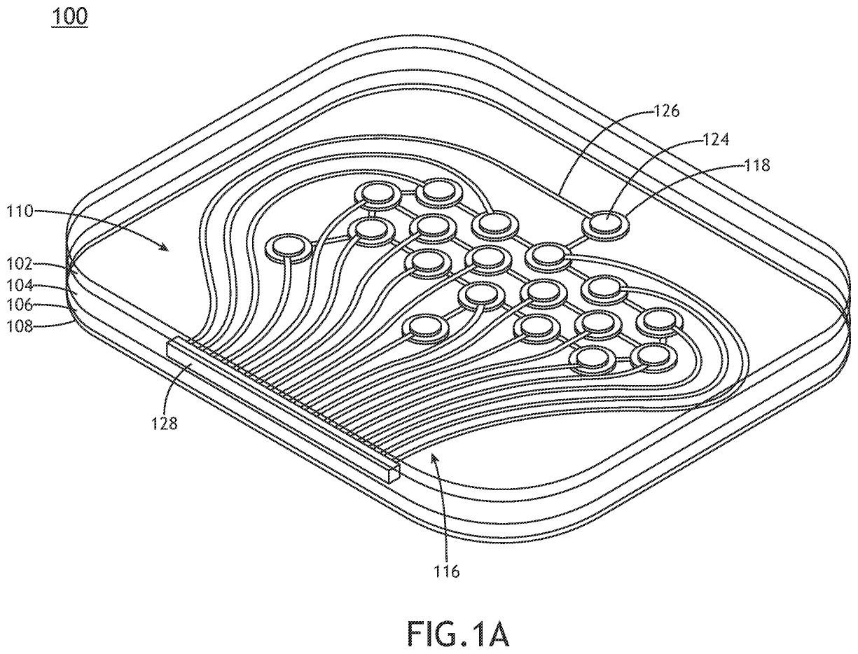

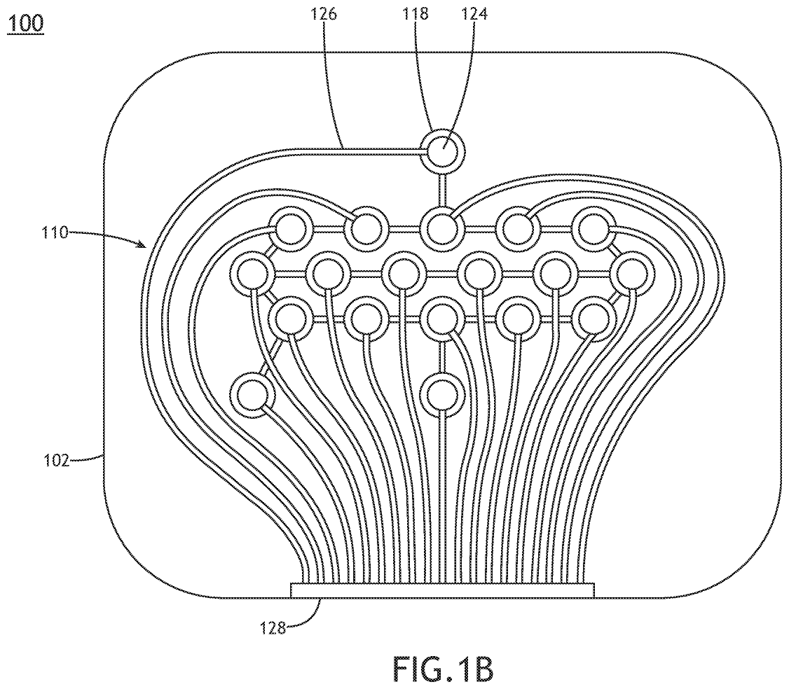

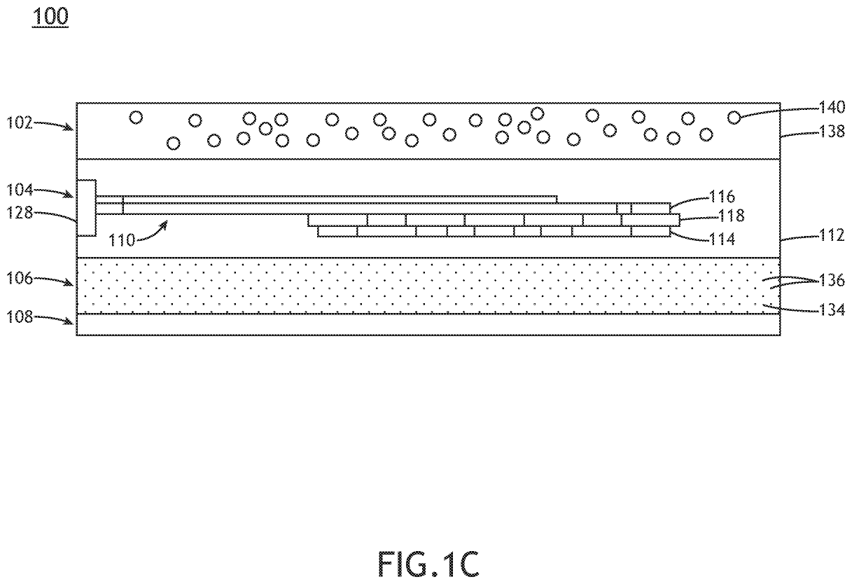

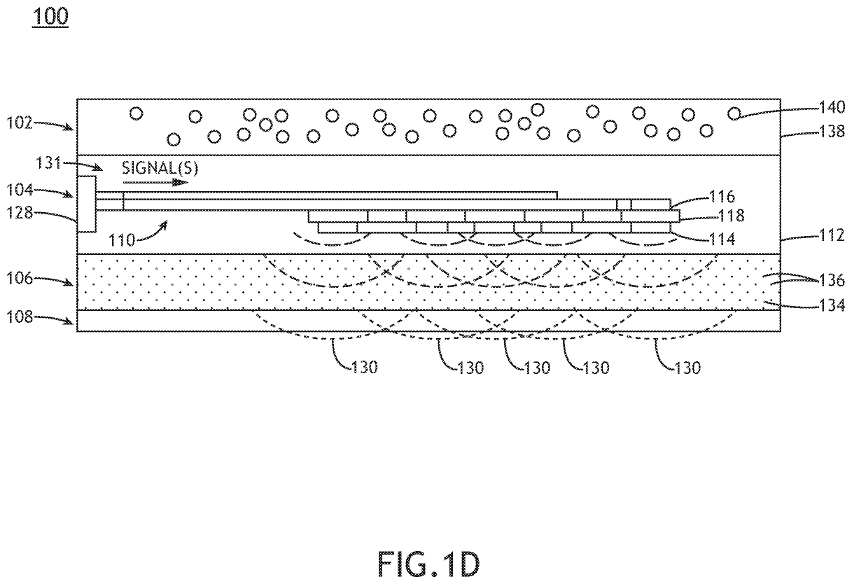

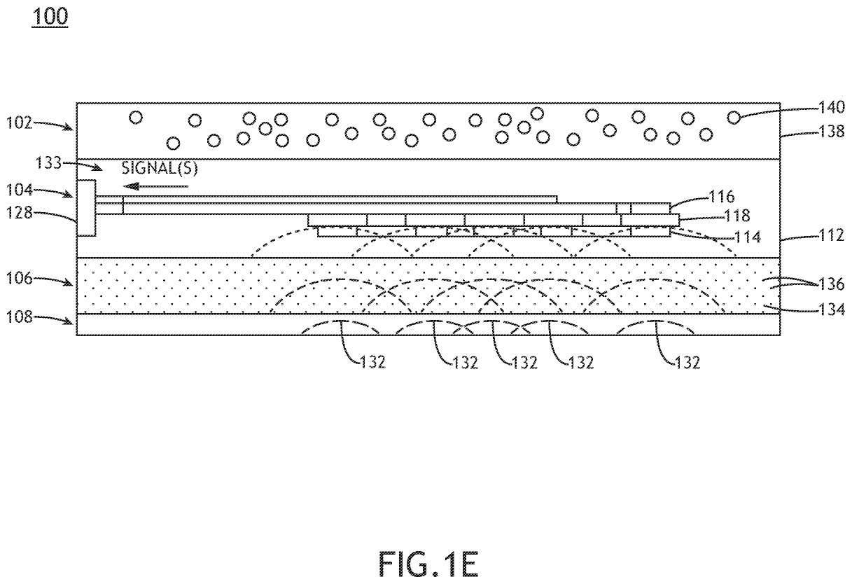

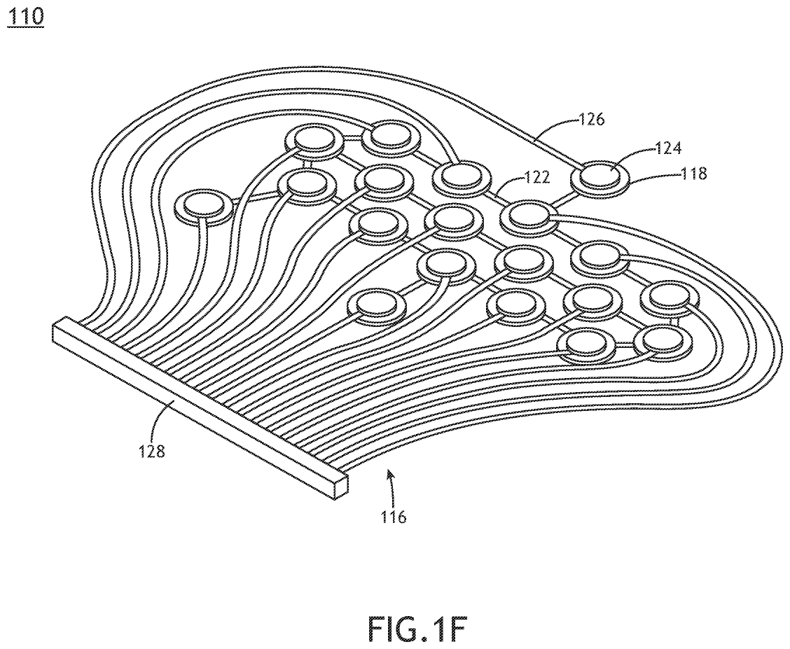

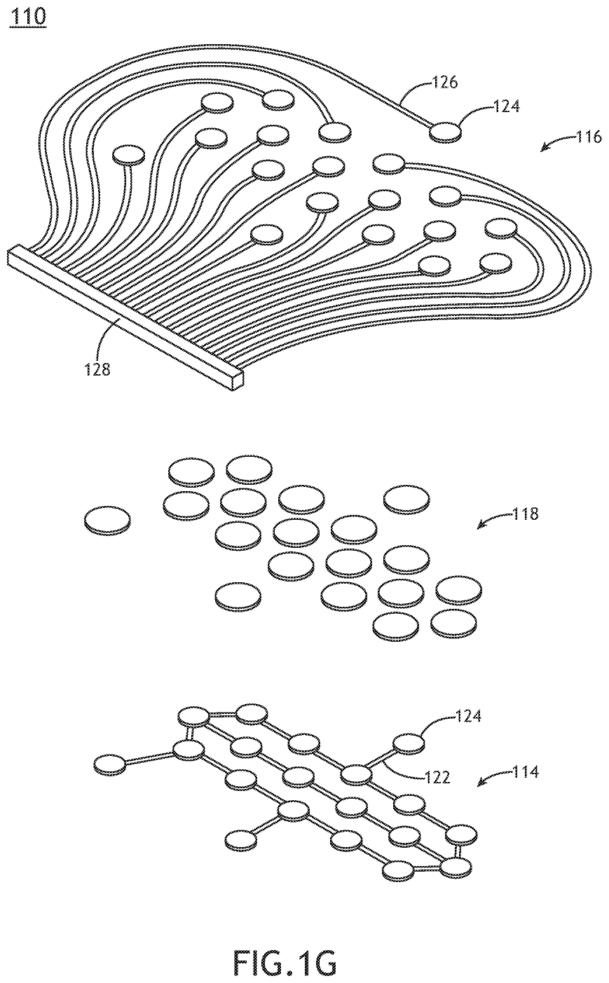

FIGS. 1A-1G illustrate a wearable ultrasound patch 100, in accordance with one or more embodiments of the present disclosure. The wearable ultrasound patch 100 may be a wearable ultrasound device, a stretchable ultrasound imaging device, a wearable transcranial doppler ultrasound (TCD) ultrasound patch, or the like. The wearable ultrasound patch 100 may include one or more layers. For example, the wearable ultrasound patch 100 may include one or more of a backing layer 102, a transducer layer 104, a matching layer 106, and/or an adhesive layer 108.

The backing layer 102 may be disposed above the transducer layer 104. The backing layer 102 may be abutted to the transducer layer 104. The backing layer 102 may be a top-most layer of the wearable ultrasound patch 100. The backing layer 102 may be a liquid metal backing layer. The backing layer 102 may include an elastomer 138 and liquid-metal microdroplets 140.

The transducer layer 104 may be disposed between the backing layer 102 and the matching layer 106. The transducer layer 104 may be abutted to the backing layer 102 and/or the matching layer 106. The transducer layer 104 may include an electrical circuit 110 and an elastomer 112. The electrical circuit 110 may include one or more of a ground plane 114, feed lines 116, and piezoelectric elements 118. The ground plane 114 may include ground electrodes 120 and/or ground traces 122. The feed lines 116 may include feed electrodes 124, feed traces 126, and/or an interface 128.

The matching layer 106 may be disposed below the transducer layer 104 and/or above the adhesive layer 108. The matching layer 106 may be disposed between the transducer layer 104 and the adhesive layer 108. The matching layer 106 may be abutted to the transducer layer 104 and/or the adhesive layer 108. The matching layer 106 may be a liquid-metal embedded elastomer (LMEE) composite. The matching layer 106 may include an elastomer 134 and liquid-metal microdroplets 136.

The adhesive layer 108 may be disposed below the matching layer 106. The adhesive layer 108 may be abutted to the matching layer 106. The adhesive layer 108 may be adhered to the matching layer 106. The adhesive layer 108 may be a bottom-most layer of the wearable ultrasound patch 100. The adhesive layer 108 may include a flexible skin adhesive, such as a double-sided medical adhesive.

The backing layer 102, the transducer layer 104, the matching layer 106, and/or the adhesive layer 108 may be conformal. For example, the backing layer 102, the transducer layer 104, the matching layer 106, and/or the adhesive layer 108 may be configured to conform to a planar surface and/or a curved surface. The backing layer 102, the transducer layer 104, the matching layer 106, and/or the adhesive layer 108 may not be rigid.

The backing layer 102, the transducer layer 104, the matching layer 106, and/or the adhesive layer 108 may be soft and/or stretchable. The backing layer 102 and/or the matching layer 106 may be soft and/or stretchable for all ranges of loadings of the liquid-metal microdroplets 136 and for all ranges of loadings of the liquid-metal microdroplets 140. The backing layer 102 and/or the matching layer 106, and/or the adhesive layer 108 may include a Shore A hardness of about 40. The backing layer 102 and/or the matching layer 106 may include an elastic modulus of between 10 kPa and 100 MPa. The backing layer 102 and/or the matching layer 106 may include an elastic modulus of between 10 kPa and 1 MPa. For example, the backing layer 102 and/or the matching layer 106 may include an elastic modulus of between 10 kPa and 160 kPa. The backing layer 102 and/or the matching layer 106 may also exhibit low hysteresis elasticity. The backing layer 102 and/or the matching layer 106 may be highly stretchable. For example, the backing layer 102 and/or the matching layer 106 may include greater than 100% strain.

The elastomer 112, the elastomer 134, and/or the elastomer 138 may be a soft material. The elastomer 112, the elastomer 134, and/or the elastomer 138 may be configured to conform to curved surfaces, such as a temple of a human body. The elastomer 112, the elastomer 134, and/or the elastomer 138 may be a silicone elastomer, also referred to as a silicone matrix. For example, the elastomer 112, the elastomer 134, and/or the elastomer 138 may include Polydimethylsiloxane (PDMS), commercially available as Sylgard™ 184 silicon elastomer, polyurethane, polyvinylsiloxane, butyl rubber, fluorosilicate, styrene butadiene, or the like.

The elastomer 112 may embed at least a portion of the electrical circuit 110. Embedding may also be referred to as encapsulating, and/or encasing. For example, the elastomer 112 may embed the ground plane 114, the piezoelectric elements 118, and a portion of the feed lines 116. For instance, the elastomer 112 may embed the feed electrodes 124, the feed traces 126, and a portion of the interface 128. A remainder of the interface 128 may not be embedded by the elastomer 112, thereby allowing for an external source to couple with the interface 128.

The elastomer 112 may be a dielectric. The elastomer 112 may insulate electric current within the electrical circuit 110, thereby preventing electric current flow from the transducer layer 104 to the backing layer 102 and/or the matching layer 106.

The elastomer 112 may be adhered to the electrical circuit 110. Air bubbles may be removed from the elastomer 112 before and/or during curing of the elastomer 112 to promote the adhesion of the elastomer 112 to the electrical circuit 110.

The piezoelectric elements 118, the ground electrodes 120, and/or the feed electrodes 124 may be planar members. The piezoelectric elements 118, the ground electrodes 120, and/or the feed electrodes 124 may be flat along a horizontal plane of the transducer layer 104. The piezoelectric elements 118, the ground electrodes 120, and/or the feed electrodes 124 may include a uniform thickness, such that the piezoelectric elements 118, the ground electrodes 120, and/or the feed electrodes 124 may be thinner than wide or long. The piezoelectric elements 118, the ground electrodes 120, and/or the feed electrodes 124 may not include any significant curvature along a horizontal plane.

The piezoelectric elements 118, the ground electrodes 120, and/or the feed electrodes 124 may include a shape, such as, but not limited to, a circular shape. The piezoelectric elements 118, the ground electrodes 120, and/or the feed electrodes 124 may include a select diameter. The diameter of the piezoelectric elements 118 may be between 8 and 25 millimeters. For example, the piezoelectric elements 118 may include a diameter of 12.7 millimeters (0.5 inches). By way of another example, the piezoelectric elements 118 may include a diameter of 18 millimeters. The diameter of the ground electrodes 120 and/or the feed electrodes 124 may be less than the diameter of the piezoelectric elements 118.

The piezoelectric elements 118 may couple the ground plane 114 and the feed lines 116. The piezoelectric elements 118 may couple the feed electrodes 124 and the ground electrodes 120 by which the ground plane 114 and the feed lines 116 may be coupled. The piezoelectric elements 118 may couple respective of the feed electrodes 124 and the ground electrodes 120. For example, each of the piezoelectric elements 118 may couple to one of the feed electrodes 124 and one of the ground electrodes 120. A ground side of the piezoelectric elements 118 may be grounded together by the ground plane 114.

The ground plane 114 may be disposed below the piezoelectric elements 118. The ground plane 114 may be a common ground to which the piezoelectric elements 118 are grounded. The ground plane 114 may couple each of the piezoelectric elements 118. The piezoelectric elements 118 may be grounded together via the ground plane 114. The ground electrodes 120 may couple to a bottom face of the piezoelectric elements 118. The ground traces 122 may couple between at least two of the ground electrodes 120, such that each of the ground electrodes 120 are coupled thereby forming the ground plane 114. For example, the ground electrodes 120 and the ground traces 122 may form the ground plane 114 in an interconnected grid.

The feed lines 116 may be disposed above the piezoelectric elements 118. The feed lines 116 may couple to individual of the piezoelectric elements 118. The feed lines 116 may be separate feed lines by which the piezoelectric elements 118 may be individually fed with current. The feed lines 116 may individually control the piezoelectric elements 118. The feed electrodes 124 may couple to a top face of the piezoelectric elements 118. The feed traces 126 may couple to individual of the feed electrodes 124.

The ground plane 114 and/or the feed lines 116 may be electrically conductive. The ground plane 114 and/or the feed lines 116 may be made of an electrically conductive material, such as, but not limited to, copper. For example, the ground electrodes 120, the ground traces 122, the feed electrodes 124, and/or the feed traces 126 may be copper.

The piezoelectric elements 118 may be bonded to the ground electrodes 120 and/or the feed electrodes 124. The piezoelectric elements 118 may be bonded to the ground electrodes 120 and/or the feed electrodes 124 by a conductive epoxy. For example, the conductive epoxy may include a conductive silver epoxy, such as 8331D commercially available from MG Chemicals™.

The ground traces 122 and/or the feed traces 126 may be soldered to the ground electrodes 120 and the feed electrodes 124, respectively. For example, the ground traces 122 and/or the feed traces 126 may be soldered to the ground electrodes 120 and the feed electrodes 124, respectively, by a low-temperature solder.

The electrical circuit 110 may be flexible. The ground plane 114 and/or the feed lines 116 may be flexible thereby enabling flexure of the electrical circuit 110. For example, the ground traces 122 of the ground plane 114 and/or the feed traces 126 of the feed lines 116 may be flexible. The ground traces 122 and/or the feed traces 126 may include a flat flex-ribbon cable, or the like, by which the ground traces 122 and/or the feed traces 126 may be flexible. The ground electrodes 120, the piezoelectric elements 118, and/or the feed electrodes 124 may or may not be flexible.

The feed lines 116 may not touch adjacent of the feed lines 116. For example, the feed electrodes 124 and/or the feed traces 126 may not touch adjacent of the feed electrodes 124 and/or adjacent of the feed traces 126. Thus, the feed lines 116 may not be grounded together. Although the feed lines 116 are described as not touching adjacent of the feed lines 116, this is not intended as a limitation of the present disclosure. It is contemplated that the feed lines 116 may be sheathed by a dielectric such that the feed lines 116 may touch adjacent of the feed lines 116 without grounding the feed lines 116 together.

The piezoelectric elements 118 may be piezoelectric transducers, piezoelectric transducer elements, ultrasound elements, ultrasound transducers, piezoelectric ultrasound transducer elements, ultrasound generating elements, ultrasound receiving elements, or the like.

The electrical circuit 110 of the transducer layer 104 may be configured to generate the emitted ultrasound waves 130 and/or receive the received ultrasound waves 132. The piezoelectric elements 118 may be configured to generate the emitted ultrasound waves 130 in response to receiving the input signals 131. The piezoelectric elements 118 may generate the emitted ultrasound waves 130 by transducing the input signals 131 to the emitted ultrasound waves 130 by vibration. The piezoelectric elements 118 may be configured to generate the output signals 133 in response to receiving the received ultrasound waves 132. The piezoelectric elements 118 may generate the output signals 133 by transducing the vibration induced by the received ultrasound waves 132 to the output signals 133. Thus, the piezoelectric elements 118 may be both ultrasound transmitters and ultrasound receivers.

The output signals 133 may include A-lines. The A-lines may be raw ultrasound data recorded during one or more pulses of the received ultrasound waves 132.

The emitted ultrasound waves 130 may pass from the piezoelectric elements 118 through the transducer layer 104, the matching layer 106 and/or the adhesive layer 108.

The received ultrasound waves 132 may be the backpropagation of the emitted ultrasound waves 130. The emitted ultrasound waves 130 may be configured to reflect from one or more external objects and backpropagate as the received ultrasound waves 132. The received ultrasound waves 132 may pass from the adhesive layer 108 through the matching layer 106 and through a portion of the transducer layer 104 to the piezoelectric elements 118.

The piezoelectric elements 118 may be configured to receive the received ultrasound waves 132. The piezoelectric elements 118 may be configured to generate output signals 133 in response to receiving the received ultrasound waves 132.

The emitted ultrasound waves 130 and/or the received ultrasound waves 132 may include a center frequency. The center frequency may be in the ultrasound range of 20 KHz to 18 MHz, or above. For example, the center frequency may include 1 MHZ, 2 MHZ, 2.225 MHz, 2.25 MHz, 2.2375 MHz, 2.2625 MHz, 2.275, MHz, 2.75 MHz, or a value therebetween.

The matching layer 106 may attenuate the emitted ultrasound waves 130 and/or the received ultrasound waves 132. The attenuation may decrease the amplitude of the emitted ultrasound waves 130 and/or the received ultrasound waves 132. The matching layer 106 may attenuate the emitted ultrasound waves 130 and/or the received ultrasound waves 132 based on an acoustic impedance (Z).

The acoustic impedance (Z) may be a resistance of matching layer 106 to the transmittance of sound waves (e.g., the emitted ultrasound waves 130 and/or the received ultrasound waves 132). Higher acoustic impedance (Z) may cause more energy of the emitted ultrasound waves 130 and/or the received ultrasound waves 132 to be lost, resulting in a weaker signal.

The acoustic impedance of the matching layer 106 may be between 1 and 7.2 MRayls. The acoustic impedance of the matching layer 106 may be between 4.8 and 7.2 MRayls. The acoustic impedance of the matching layer 106 may be between 5.6 and 5.8 MRayl. The acoustic impedance of the matching layer 106 may be between 5.73 and 5.75. For example, the acoustic impedance of the matching layer 106 may be 5.74 MRayl. By way of another example, the acoustic impedance of the matching layer 106 may be 5.745 MRayls.

The acoustic impedance (Z) of the matching layer 106 may be determined by the density (p) of the material and the speed of sound (c) through the material, as defined by the following formula:

$$Z = ρ \cdot c$$

The liquid-metal microdroplets 136 may be dispersed within the elastomer 134. The liquid-metal microdroplets 136 may be uniformly or non-uniformly dispersed within the elastomer 134. The elastomer 134 may be a silicone-based elastomer with a relatively low density. The density of the liquid-metal microdroplets 136 may be larger than the density of the elastomer 134. The liquid-metal microdroplets 136 may thereby increase the density of the matching layer 106 without significantly impacting the elastic modulus of the matching layer 106.

The speed of sound through the matching layer 106 may be proportional to the volume loading of the liquid-metal microdroplets 136 for all diameters of the liquid-metal microdroplets 136. The increase in the speed of sound may be predicted by Wood's model, by the following formula:

$${c}_{Wood} = \frac{1}{\sqrt{[ ( 1 - ϕ ) {ρ}_{m} + ϕ {ρ}_{f} ] \cdot [ ( 1 - ϕ ) {χ}_{m} + ϕ {χ}_{f} ]}}$$

Where, the theoretical speed of sound (c_{Wood}) is calculated from the LM density (ρ_{f}), elastomer matrix density (ρₘ), volume loading (ϕ) of the liquid-metal microdroplets 136, compressibility (Xₘ) of the elastomer 134, and compressibility (X_{f}) of the liquid-metal microdroplets 136. Thus, the acoustic impedance of the matching layer 106 may be proportional to the volume loading of the liquid-metal microdroplets 136 and/or proportional to the diameter of the liquid-metal microdroplets 136. The acoustic impedance of the matching layer 106 may be tuned by varying the volume loading and/or diameter of the liquid-metal microdroplets 136.

The liquid-metal microdroplets 136 may include a select diameter. The diameter of the liquid-metal microdroplets 136 may be on the micrometer scale and/or a nanometer scale. The liquid-metal microdroplets 136 may include a diameter of 10 nanometers, 100 nanometers, 1 micrometer, 10 micrometers, 20 micrometers, 100 micrometers, 500 micrometers, or a value therebetween. The liquid-metal microdroplets 136 may include a diameter between 10 nanometers and 500 micrometers. The liquid-metal microdroplets 136 may include a diameter between 100 nanometers and 100 micrometers. The liquid-metal microdroplets 136 may include a diameter between 100 nanometers and 1 micrometer. The liquid-metal microdroplets 136 may include a diameter between 1 and 20 micrometers. The liquid-metal microdroplets 136 may include a diameter between 1 and 10 micrometers.

The liquid-metal microdroplets 136 may include a selected volume loading (ϕ). The volume loading may also be referred to as a volume fraction. The volume loading may be a volume of the liquid-metal microdroplets 136 divided by the sum of the volume of the liquid-metal microdroplets 136 and the elastomer 134. The liquid-metal microdroplets 136 may include a volume loading between 10% and 80%. For example, the liquid-metal microdroplets 136 may include a volume loading between 10% and 50%. By way of another example, the liquid-metal microdroplets 136 may include a volume loading between 50% and 70%. By way of another example, the liquid-metal microdroplets 136 may include a volume loading between 70% and 80%.

In embodiments, the liquid-metal microdroplets 136 may include a diameter of 1 micrometer and a volume loading of 50%.

The liquid-metal microdroplets 136 may include a gallium-based liquid-metal (Ga-based LM). The liquid-metal microdroplets 136 may include a eutectic gallium-based liquid-metal. The eutectic gallium-based liquid-metal may be selected due to a combination of high electrical and thermal conductivity, low viscosity, and nontoxic characteristics. The eutectic gallium-based liquid-metal may include eutectic gallium-indium (EGaIn), a eutectic alloy of gallium, indium, and tin (e.g., Galinstan™), or the like. The EGaIn may include 75 wt. % Ga and 25 wt. % In. The EGaIn may include a density (p) of 6.25 g/ml. The eutectic alloy of gallium, indium, and tin may include 68.5 wt. % Ga, 21.5 wt. % In, and 10 wt. % Sn.

The matching layer 106 may include a selected thickness. For example, the thickness of the matching layer 106 may be 200 micrometers.

The backing layer 102 may be configured to attenuate one or more backwards-traveling portions of one or more ultrasound waves from the transducer layer 104. The received ultrasound waves 132 may pass through the transducer layer 104 up to the backing layer 102. The backing layer 102 may attenuate the received ultrasound waves 132, thereby preventing the propagation of the received ultrasound waves 132 through the backing layer 102. The backing layer 102 may provide a significant effect on shaping the received ultrasound waves 132. The backing layer 102 may include an acoustic impedance which is larger than the matching layer 106.

The liquid-metal microdroplets 140 of the backing layer 102 may include a select diameter. The diameter of the liquid-metal microdroplets 140 may be much larger than the diameter of the liquid-metal microdroplets 136. In this regard, the backing layer 102 may include a much higher acoustic attenuation than the matching layer 106.

The interface 128 may be configured to transmit the input signals 131 to the piezoelectric elements 118 and/or receive the output signals 133 from the piezoelectric elements 118. The interface 128 may be configured to transmit the input signals 131 to the piezoelectric elements 118 and/or receive the output signals 133 from the piezoelectric elements 118 via the feed lines 116. The wearable ultrasound patch 100 may be configured to receive the input signals 131 from an external source and/or transmit the output signals 133 to the external source via the interface 128.

The interface 128 may include a wired interface and/or a wireless interface. For example, the interface may include a wireline-interface (e.g., wired-ribbon interface, and the like). By way of another example, the interface 128 may include a wireless-interface (e.g., Bluetooth, WiFi, and the like). It is contemplated that the wireless interface may enable continuous monitoring of the output signals 133.

The piezoelectric elements 118 may be independently addressable from the feed lines 116. Independently addressing the piezoelectric elements 118 may enabling acoustic beamforming of the emitted ultrasound waves 130. The wearable ultrasound patch 100 may be configured to perform the acoustic beamforming of the piezoelectric elements 118 by receiving the input signals 131 at separate phases. For example, each of the piezoelectric elements 118 may be configured to receive a separate one of the input signals 131 from the feed lines 116 and generate a separate one of the emitted ultrasound waves 130. The emitted ultrasound waves 130 may then interfere (e.g., constructively and/or destructively interfere). The phase of the input signals 131 may be controlled to control the interference of the emitted ultrasound waves 130 thereby beamforming the emitted ultrasound waves 130.

The piezoelectric elements 118 may be arranged in an array. The array may include at least two of the piezoelectric elements 118. The array may include a planar array. The planar array may include at least three of the piezoelectric elements 118 which are co-planar and are not co-linear.

One example of the array of the piezoelectric elements 118 may include eighteen of the piezoelectric elements 118. Each of the eighteen of the piezoelectric elements 118 may be coupled to one of eighteen of the feed lines 116. In this example, the array of the piezoelectric elements 118 is a five-row array with a first row, second row, third row, fourth row, and a fifth row of the piezoelectric elements 118. One of the piezoelectric elements 118 may be in the first row, five of the piezoelectric elements 118 may be in the second row, six of the piezoelectric elements 118 may be in the third row, five of the piezoelectric elements 118 may be in the fourth row, and two of the piezoelectric elements 118 may be in the fifth row.

The first piezoelectric element in the first row (row1, column1), the third piezoelectric element in the second row (row2, column3), the third piezoelectric element in the fourth row (row4, column3), and the second piezoelectric element in the fifth row (row5, column2) may be aligned.

Each of the piezoelectric elements in the second row may be aligned with respective elements in the fourth row. For example, the first piezoelectric element in the second row (row2, column1) and the first piezoelectric element in the fourth row (row4, column1), the second piezoelectric element in the second row (row2, column2) and the second piezoelectric element in the fourth row (row4, column2), the third piezoelectric element in the second row (row2, column3) and the third piezoelectric element in the fourth row (row4, column3), the fourth piezoelectric element in the second row (row2, column4) and the fourth piezoelectric element in the fourth row (row4, column4), the fifth piezoelectric element in the second row (row2, column5) and the fifth piezoelectric element in the fourth row (row4, column5), respectively, may be aligned.

The first piezoelectric element in the third row (row3, column1) and the first piezoelectric element in the fifth row (row5, column1) may be aligned.

The second row, third row, and fourth row of the piezoelectric elements 118 may form a lattice. For example, the lattice may be a triangular lattice.

The shape of the piezoelectric elements 118 in the array may be selected so that an area covered by the piezoelectric elements 118 may be used for monitoring a middle cerebral artery (MCA) by beamforming the emitted ultrasound waves 130. The exemplary configuration of the array may enable monitoring the middle cerebral artery. The exemplary configuration of the array was experimentally determined to enable monitoring the middle cerebral artery.

Although the wearable ultrasound patch 100 is described as including the adhesive layer 108, this is not intended as a limitation of the present disclosure. It is contemplated that the matching layer 106 may be a bottom-most layer of the wearable ultrasound patch 100. The matching layer 106 may perform one or more functions of the adhesive layer 108. For example, the matching layer 106 may include one or more adhesive properties by which the wearable ultrasound patch 100 may adhere to one or more surfaces. In this regard, the matching layer 106 may be a combined matching and adhesive layer.

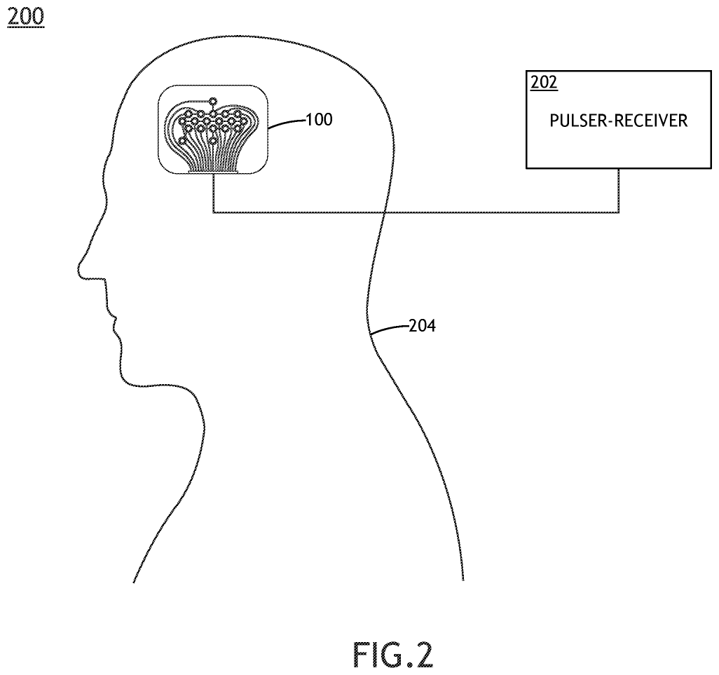

FIG. 2 illustrates a system 200, in accordance with one or more embodiments of the present disclosure. The system 200 may include one or more of the wearable ultrasound patch 100, a pulser-receiver 202, and/or a patient 204.

The pulser-receiver 202 may include, but is not limited to, a Panametrics™ 500 PR pulser-receiver.

The pulser-receiver 202 may include a user interface. The user interface may include, but is not limited to, one or more desktops, laptops, tablets, and the like. The user interface may include a display used to display the output signals 133. The display of the user interface may include any display known in the art. For example, the display may include, but is not limited to, a liquid crystal display (LCD), an organic light-emitting diode (OLED) based display, or a CRT display.

The wearable ultrasound patch 100 may be connected to the pulser-receiver 202. For example, the interface 128 of the wearable ultrasound patch 100 may be connected to the pulser-receiver 202. The pulser-receiver 202 may be configured to transmit the input signals 131 to the interface 128 and receive the output signals 133 from the interface 128. The pulser-receiver 202 may cause the piezoelectric elements 118 to emit the emitted ultrasound waves 130 by transmitting the input signals 131 to the interface 128.

The wearable ultrasound patch 100 may be wearable by the patient 204. It is contemplated that the wearable ultrasound patch 100 may be worn for an extended period. The wearable ultrasound patch 100 may conform to one or more surfaces of the patient 204. For example, the wearable ultrasound patch 100 may conform to a temple of the patient 204. The wearable ultrasound patch 100 may be adhered to the patient 204. For example, the wearable ultrasound patch 100 may be adhered to the temple of the patient 204 by the adhesive layer 108.

The patient 204 may be a post-operation subarachnoid hemorrhage (SAH) patient that is at high-risk for developing a vasospasm, a potentially fatal condition in which there is persistent contraction of the blood vessels thereby decreasing blood flow. After traumatic brain injury or initial stroke, there is a time window in which a secondary stroke could possibly occur with devastating consequences for the patient. This secondary stroke could potentially be predicted and prevented by frequent periodic monitoring of the cerebral blood flow of the patient 204.

The patient 204 may include skin. The emitted ultrasound waves 130 may be sent through the skin to tissue of the patient 204 disposed below the skin. The emitted ultrasound waves 130 may reflect from the tissue of the patient 204 as the received ultrasound waves 132.

The matching layer 106 may be disposed between the transducer layer 104 and the patient 204. The matching layer 106 may improve the transmittance of the emitted ultrasound waves 130 and/or the received ultrasound waves 132 between the piezoelectric elements 118 and the skin. The piezoelectric elements 118 and the skin may have different acoustic impedances. The matching layer 106 may be configured to match the acoustic impedance of the piezoelectric elements 118 with the acoustic impedance of the skin of the patient 204. The optimal acoustic impedance of the matching layer 106 may be found using the geometric mean of the two surrounding materials. The piezoelectric elements 118 may include an acoustic impedance (Z1) of 22 MRayl. The skin may include an acoustic impedance (Z2) of 1.5 MRayl. The optimal acoustic impedance of the matching layer 106 (Zm) may be the geometric mean of the piezoelectric material (Z1=22 MRayl) and the imaged medium (Z2=1.5 MRayl). For the wearable ultrasound patch, the optimal acoustic impedance was determined to be 5.74 MRayl.

The pulser-receiver 202 may cause the wearable ultrasound patch 100 to collect data from the patient 204. The wearable ultrasound patch 100 may continuously measure blood flow velocity, blood pressure, and the like. The data may be measured non-invasively. The wearable ultrasound patch 100 and/or the pulser-receiver 202 may be configured to perform digital signal processing on the output signals 133 to produce real-time cerebral blood flow velocity measurements. For example, the output signals 133 may include a Doppler spectrum which may show the increase and decrease in blood flow velocity in the brain as the heart pumps the blood.

The wearable ultrasound patch 100 may be transcranial Doppler ultrasound (TCD). The pulser-receiver 202 may continuously measure blood flow velocity in major cerebral arteries, such as the middle cerebral artery. In particular, the wearable ultrasound patch 100 may continuously measure blood flow velocity through the middle cerebral artery (MCA) that can be assessed from the temple of the patient 204.

The piezoelectric elements 118 may be connected to the pulser-receiver 202. The piezoelectric elements 118 may be independently addressable by the pulser-receiver 202. The pulser-receiver 202 may be configured to cause the piezoelectric elements 118 to beamform the emitted ultrasound waves 130 by controlling a phase of the interface 128. The acoustic beamforming of the emitted ultrasound waves 130 may enable the wearable ultrasound patch 100 to locate a middle cerebral artery (MCA). For example, the wearable ultrasound patch 100 may locate the middle cerebral artery while the wearable ultrasound patch 100 is adhered to the temple of the patient 204. For instance, the pulser-receiver 202 may use the array of the piezoelectric elements 118 to identify the cerebral blood flow velocity without the need to manually reposition the wearable ultrasound patch 100 on the temple of the patient 204.

The wearable ultrasound patch 100 and/or the pulser-receiver 202 may be configured to detect a change in the blood flow velocity of the patient 204 from the output signals 133. For example, the wearable ultrasound patch 100 and/or the pulser-receiver 202 may detect the change in the blood flow velocity thereby indicating a symptom of early stroke. The strokes may include ischemic strokes, where blood clots or other particles block normal blood flow to the brain. The wearable ultrasound patch 100 and/or the pulser-receiver 202 may generate an alert in response to detecting the change in the blood flow velocity. For example, the wearable ultrasound patch 100 and/or the pulser-receiver 202 may generate an alert for a medical professional of a potential abnormality in the brain before the abnormality becomes harmful or fatal.

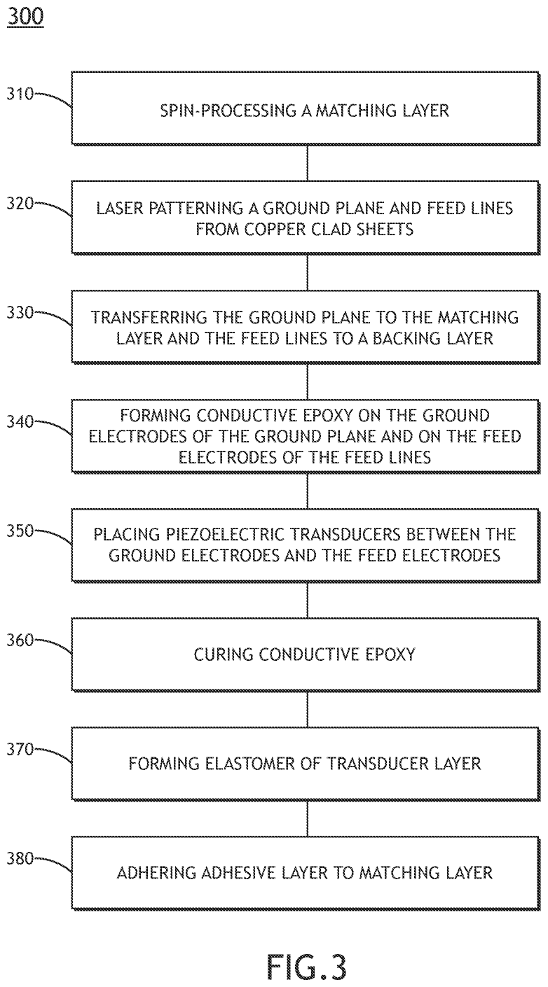

FIG. 3 illustrates a flow diagram of a method 300, in accordance with one or more embodiments of the present disclosure. The method may also be referred to as a process of manufacturing the wearable ultrasound patch 100. The embodiments and the enabling technologies described previously herein in the context of the wearable ultrasound patch 100 should be interpreted to extend to the method 300. It is further noted, however, that the method 300 is not limited to the architecture of the wearable ultrasound patch 100.

In a step 310, a matching layer may be spin-processed. For example, the matching layer 106 may be spin-processed. The matching layer 106 may be spin-processed to a desired thickness. Spin-processing the matching layer 106 may be further understood with reference to the method 500.

In a step 320, a ground plane and feed lines may be laser patterned from one or more copper clad sheets. For example, the ground plane 114 and feed lines 116 may be laser patterned from one or more copper clad sheets. The one or more copper clad sheets may include a ½ oz flexible copper clad sheet, commercially available as FR8510R from DuPont™. The one or more copper clad sheets may be supported by a sacrificial elastomer substrate during laser patterning. The ground plane 114 the feed lines 116 may be laser patterned utilizing an ultraviolet (UV) laser micromachining system, such as a scanner-guided laser.

After laser patterning, debris may be cleaned from the surface of the ground electrodes 120 and/or the feed electrodes 124. The debris may be cleaned with isopropanol or the like. The excess copper of the copper clad sheets may be removed from the elastomer substrate, leaving the ground plane 114 and the feed lines 116.

In a step 330, the ground plane may be transferred to the matching layer and the feed lines are transferred to a backing layer. For example, the ground plane 114 may be transferred to the matching layer 106 and the feed lines 116 may be transferred to the backing layer 102. The ground plane 114 may be transferred to the matching layer 106 and/or the feed lines 116 may be transferred to the backing layer 102 using a water-soluble tape, such as Aquasol™.

In a step 340, a conductive epoxy may be formed on the ground electrodes of the ground plane and on the feed electrodes of the feed lines. For example, a conductive epoxy may be formed on the ground electrodes 120 of the ground plane 114 and on the feed electrodes 124 of the feed lines 116. The conductive epoxy may include a conductive silver epoxy. The conductive epoxy may be formed on the ground electrodes 120 of the ground plane 114 by placing a first stencil mask over the ground plane 114, the first stencil mask including holes aligned with the ground electrodes 120, and blade coating the conductive epoxy onto the ground electrodes 120. The ground traces 122 and/or matching layer 106 may not be coated with the conductive epoxy by the first stencil mask covering the ground traces 122 and/or matching layer 106. The first stencil mask may then be removed. Similarly, the conductive epoxy may be formed on the feed electrodes 124 of the feed lines 116 by placing a second stencil mask over the feed lines 116, the second stencil mask including holes aligned with the feed electrodes 124, and blade coating the conductive epoxy onto the feed electrodes 124.

In a step 350, piezoelectric transducers may be placed between the ground electrodes and the feed electrodes. For example, the piezoelectric elements 118 may be placed between the ground electrodes 120 and the feed electrodes 124. The piezoelectric elements 118 may be sandwiched between the ground electrodes 120 and the feed electrodes 124 with the conductive epoxy as a barrier disposed therebetween.

In a step 360, the conductive epoxy may be cured to bond the piezoelectric elements between the ground electrodes and the feed electrodes thereby forming the electrical circuit 110 of the transducer layer 104. For example, the conductive epoxy may be cured to bond the piezoelectric elements 118 between the ground electrodes 120 and the feed electrodes 124 thereby forming the electrical circuit of a transducer layer. The conductive epoxy may be cured by placing the matching layer 106, the ground plane 114, the piezoelectric elements 118, the feed lines 116, and the backing layer 102 in an oven at 80° C. for 40 minutes to cure the conductive epoxy.

In a step 370, an elastomer of the transducer layer may be formed. For example, the elastomer 112 of the transducer layer 104 may be formed. The elastomer 112 of the transducer layer 104 may be formed by encapsulating the electrical circuit 110 with the PDMS and cured. The PDMS may be cured at 80° C. for 1 hour in a pressure chamber at 40 PSIG.

In a step 380, an adhesive layer may be adhered to the matching layer. For example, the adhesive layer 108 may be adhered to the matching layer 106.

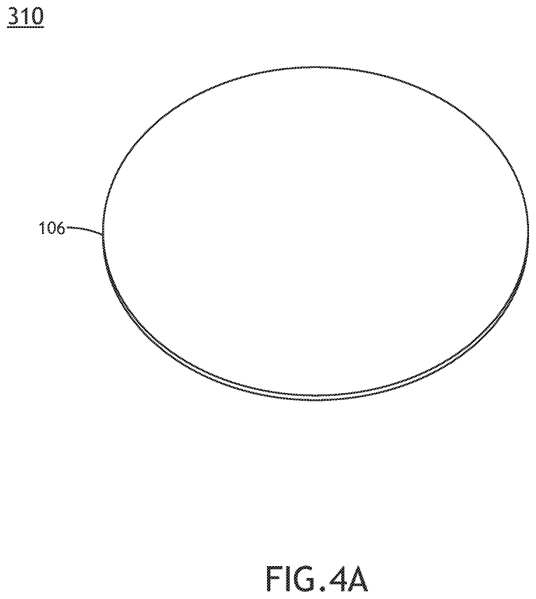

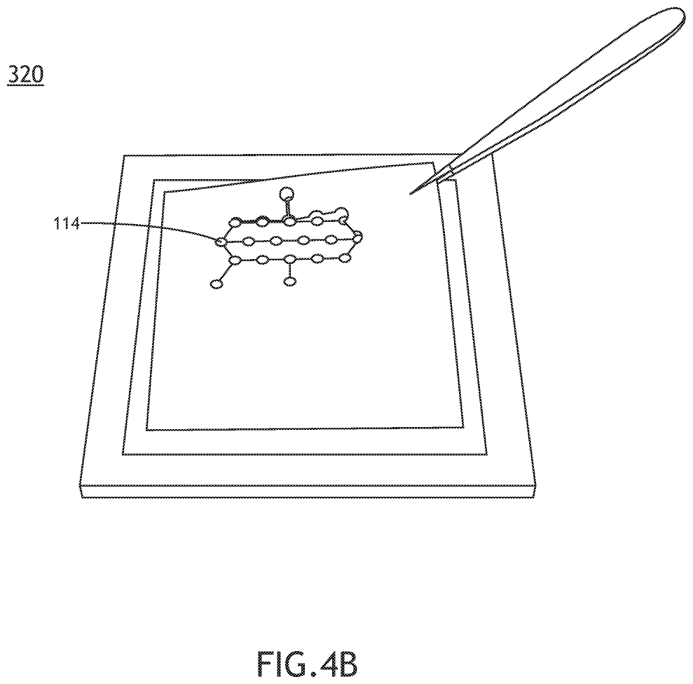

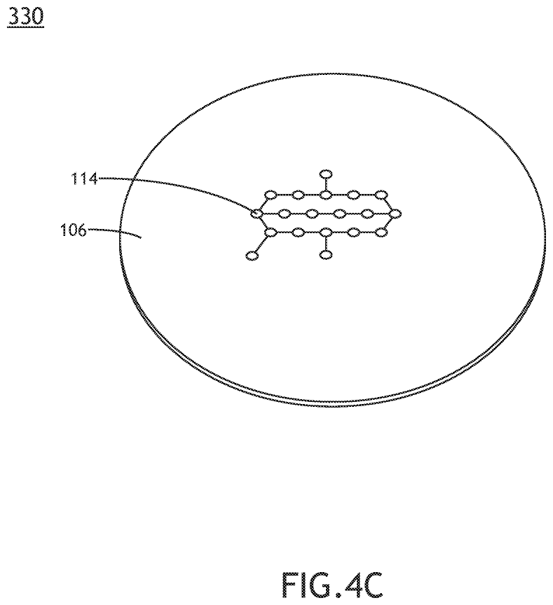

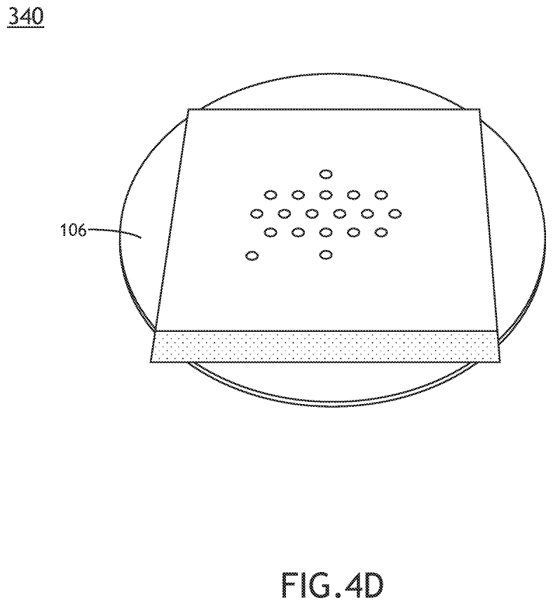

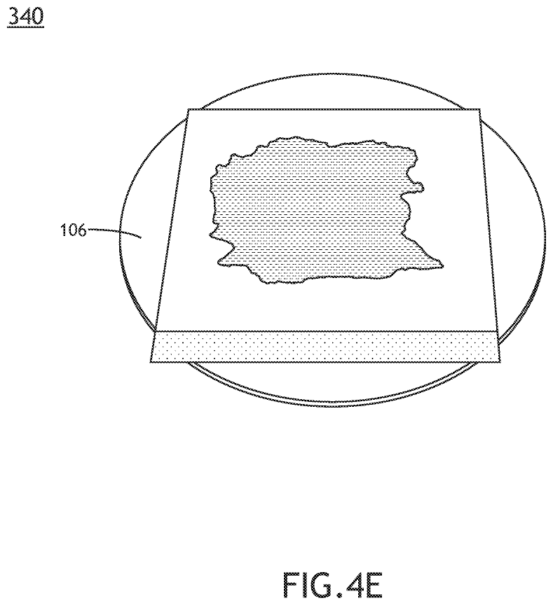

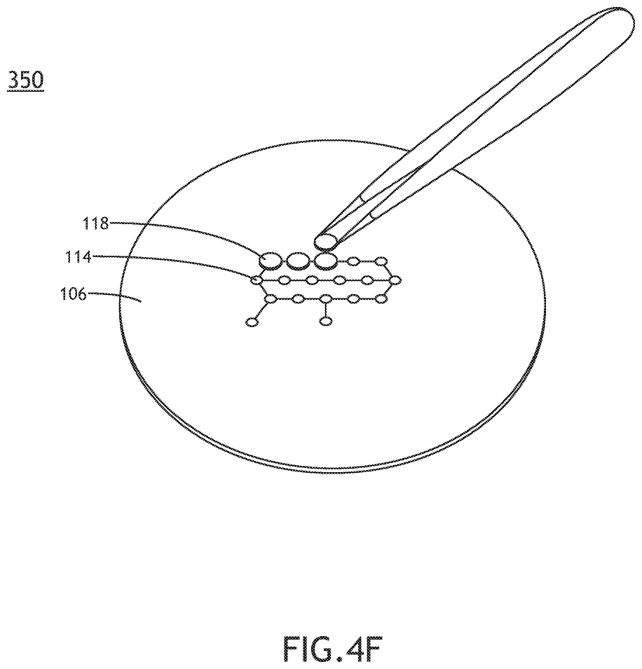

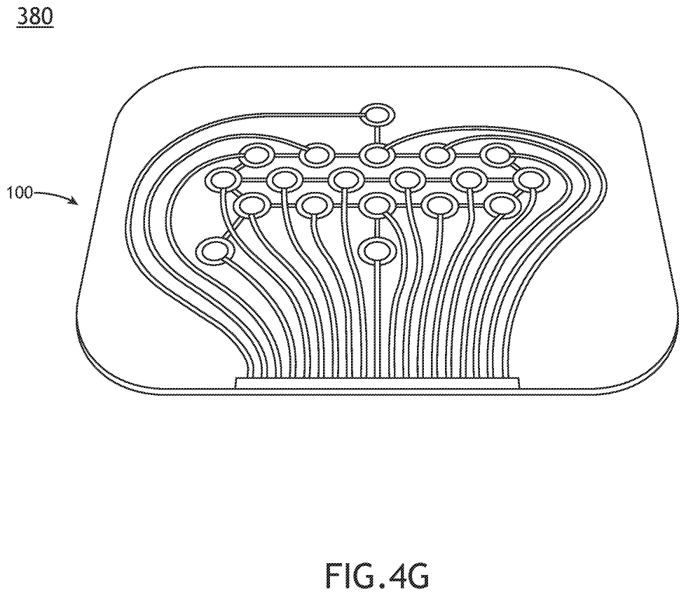

FIGS. 4A-4G illustrate one or more of the steps of the method 300. FIG. 4A illustrates the matching layer 106 after spin-processing, in accordance with one or more embodiments of the present disclosure. FIG. 4B illustrates the ground plane 114 during removal of the excess copper of the copper clad sheets, in accordance with one or more embodiments of the present disclosure. FIG. 4C illustrates the ground plane 114 after being transferred onto the matching layer 106, in accordance with one or more embodiments of the present disclosure. FIGS. 4D-4E illustrates coating the conductive epoxy onto the ground electrodes 120 of the ground plane 114 using the first stencil mask, in accordance with one or more embodiments of the present disclosure. FIG. 4F illustrates placing the piezoelectric elements 118 onto the ground electrodes 120 of the ground plane 114 before the piezoelectric elements 118 are sandwiched between the ground plane 114 and the feed lines 116, in accordance with one or more embodiments of the present disclosure. FIG. 4G illustrates the wearable ultrasound patch, in accordance with one or more embodiments of the present disclosure.

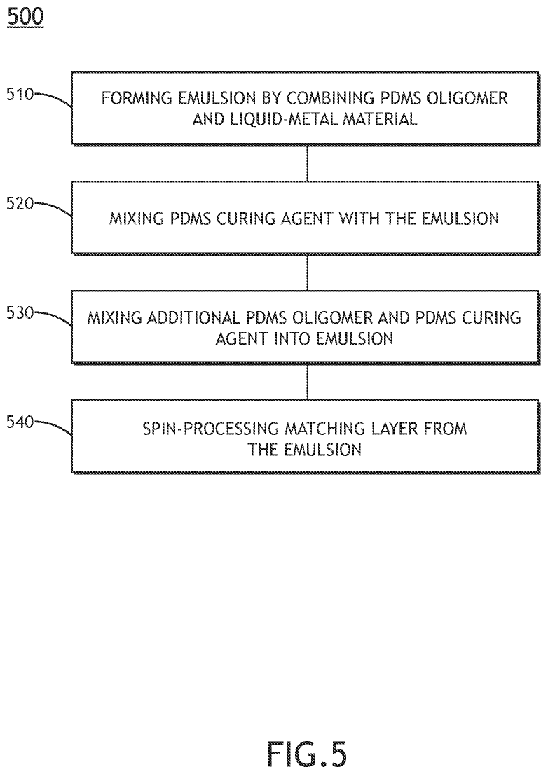

FIG. 5 illustrates a flow diagram of a method 500, in accordance with one or more embodiments of the present disclosure. The method may also be referred to as a process of spin-processing the matching layer 106. The embodiments and the enabling technologies described previously herein in the context of the matching layer 106 and the step 310 should be interpreted to extend to the method 500. It is further noted, however, that the method 500 is not limited to the architecture of the matching layer 106 and the step 310.

In a step 510, a PDMS oligomer may be combined with a liquid-metal material to form an emulsion. The PDMS oligomer may be combined with the liquid-metal material at a 0.9:1 volume ratio. The emulsion may include the liquid-metal microdroplets 136. For example, the PDMS oligomer may be combined with the liquid-metal material to form the emulsion by mixing the PDMS oligomer with the liquid-metal material using an overhead mixer at 2,200 RPM for 20 minutes, which results in the liquid-metal microdroplets 136 with a diameter of approximately 1 micrometer.

In a step 520, a PDMS curing agent may be mixed with the emulsion. The PDMS curing agent may be added to the emulsion at a 10:1 PDMS oligomer to PDMS curing agent ratio. The emulsion with the PDMS curing agent may be mixed and/or degassed. For example, the emulsion with the PDMS curing agent may be mixed and/or degassed in a planetary mixer at 1,000 RPM for 1 minute.

In a step 530, additional PDMS oligomer and additional PDMS curing agent may be mixed with the emulsion. The additional PDMS oligomer and additional PDMS curing agent may be mixed with the emulsion dilute the emulsion to an appropriate volume loading of the liquid-metal microdroplets 136. For example, to achieve volume loadings of the liquid-metal microdroplets 136 of less than 50%, additional PDMS oligomer/curing agent may be added to the emulsion thereby dilute the emulsion to the appropriate volume loading.

In a step 540, a matching layer may be spin-processed from the emulsion. For example, the matching layer 106 may be spin-processed from the emulsion. The matching layer 106 may be spin-processed to a select thickness and cured at 100° C. for 1 hour. For example, the matching layer 106 may be spin-processed to a thickness of 200 micrometers.

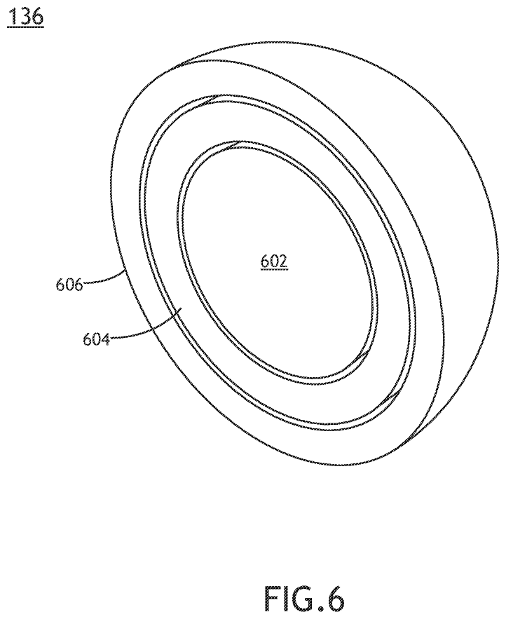

FIG. 6 illustrates a cross-section view of the liquid-metal microdroplets 136, in accordance with one or more embodiments of the present disclosure. The acoustic impedance of the matching layer 106 may be tuned by varying the density of the liquid-metal microdroplets 136. The density of the liquid-metal microdroplets 136 may be varied by introducing fillers into the liquid-metal microdroplets 136. The fillers may increase the acoustic impedance of the matching layer 106. The fillers may include but are not limited to tungsten (W). The liquid-metal microdroplets 136 may include one or more of a core 602, an adhesive shell 604, and/or a liquid-metal shell 606.

The core 602 may be made of a select material. The material from which the core 602 is made may be a metal. For example, the core 602 may include tungsten (W), tungsten carbide (WC), aluminum (Al), copper (Cu), iron (Fe), nickel (Ni), silver (Ag), alloys thereof, or other metallic microparticles. For instance, the core 602 may be a tungsten core made of tungsten microparticles. By way of another instance, the core 602 may be a tungsten carbide core made of tungsten carbide microparticles.

The core 602 may be disposed at a core of the liquid-metal microdroplets 136.

The adhesive shell 604 may trap the core 602. For example, the adhesive shell 604 may be coated onto the core 602. The adhesive shell 604 may include polydopamine (PDA). Thus, the core 602 and the adhesive shell 604 may be referred to as W@PDA.

For example, the polydopamine (PDA) may include the following chemical formula:

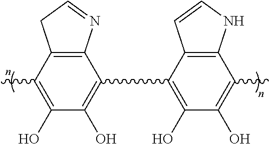

By way of another example, the polydopamine (PDA) may include the following chemical formula:

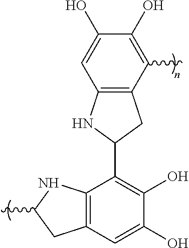

The adhesive shell 604 may be disposed between the core 602 and the liquid-metal shell 606. The adhesive shell 604 may adhere the liquid-metal shell 606 to the core 602. The liquid-metal shell 606 may trap the adhesive shell 604. For example, the liquid-metal shell 606 may be coated onto the adhesive shell 604.

The liquid-metal shell 606 may be an alloy of a first metal and the eutectic gallium-based liquid-metal. The first metal may include silver (Ag), copper, nickel, gold, tin, or the like. The eutectic gallium-based liquid-metal may include eutectic gallium-indium (EGaIn), a eutectic alloy of gallium, indium, and tin (e.g., Galinstan™), or the like. For example, the core 602 may be tungsten, the adhesive shell 604 may be polydopamine, and/or the liquid-metal shell 606 may be an alloy of silver (Ag) and the EGaIn. Thus, the liquid-metal microdroplets 136 may be W@PDA-(Ag+EGaIn) microdroplets.

Although the liquid-metal microdroplets 136 are described as including the adhesive shell 604, this is not intended as a limitation of the present disclosure. It is contemplated that the adhesive shell 604 may not be needed for all combinations of the core 602 and the liquid-metal shell 606. For example, Nickel-coated tungsten microdroplets may be commercially available as SP-1303 from Heeger Materials™, where the Nickel may be alloyed with the eutectic gallium-based liquid-metal to form the liquid-metal microdroplets 136 including the core 602 and the liquid-metal shell 606 without the adhesive shell 604.

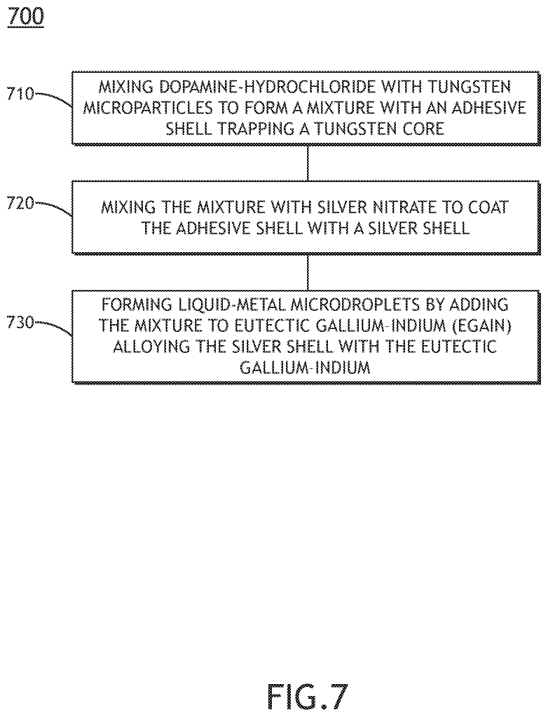

FIG. 7 illustrates a flow diagram of a method 700, in accordance with one or more embodiments of the present disclosure. The method may also be referred to method of introducing tungsten into the liquid-metal microdroplets 136. The embodiments and the enabling technologies described previously herein in the context of the liquid-metal microdroplets 136 should be interpreted to extend to the method 700. It is further noted, however, that the method 700 is not limited to the architecture of the liquid-metal microdroplets 136.

In a step 710, dopamine-hydrochloride may be mixed with tungsten microparticles to form a mixture with an adhesive shell trapping a tungsten core. For example, dopamine-hydrochloride may be mixed with tungsten microparticles to form a mixture with the adhesive shell 604 trapping the core 602. The dopamine-hydrochloride may include a pH of 8.5. The dopamine-hydrochloride may oxidize and polymerize during mixing into the polydopamine of which the adhesive shell 604 comprises. The dopamine-hydrochloride may be mixed with the tungsten microparticles at 350 RPM for 24 hours.

In a step 720, the mixture with the tungsten core and the adhesive shell may be mixed with silver nitrate (AgNO3) to coat the adhesive shell with a silver shell. For example, the mixture with the core 602 and the adhesive shell 604 may be mixed with silver nitrate (AgNO3) to coat the adhesive shell 604 with the silver shell. The mixture may be mixed at 125 RPM for 1.5 hours. A diameter of the droplets within the mixture may be 20 micrometers after mixing the silver nitrate.

In a step 730, liquid-metal microdroplets may be formed by adding the mixture to eutectic gallium-indium (EGaIn) alloying the silver shell with the eutectic gallium-indium. For example, the liquid-metal microdroplets 136 may be formed by adding the mixture to eutectic gallium-indium (EGaIn) alloying the silver shell with the eutectic gallium-indium.

The silver and eutectic gallium-indium may also facilitate oxide-mediating mixing for a high density, non-dilute mixture. The W@PDA-(Ag+EGaIn) microdroplets may include a density which is greater than the EGaIn. For example, the EGaIn may include a density of 6.25 grams per cubic centimeter and the W@PDA-(Ag+EGaIn) microdroplets may include a density greater than 6.25 grams per cubic centimeter. Air pockets formed by oxide-mediated mixing can be filled by EGaIn following several rounds of dilutions and hand-mixing, as the density of these mixtures ultimately surpasses that of pure EGaIn. Pure EGaIn may have an acoustic impedance of 16.875 MRayls, while the tungsten and EGaIn dilutions were determined to have acoustic impedances closer to 21.5 MRayls.

The W@PDA-(Ag+EGaIn) microdroplets may be dispersed within the elastomer 134 to form the matching layer 106. For example, the W@PDA-(Ag+EGaIn) microdroplets may be dispersed within the elastomer 134 using the method 500. Thus, the liquid-metal microdroplets 136 may include the W@PDA-(Ag+EGaIn) microdroplets.

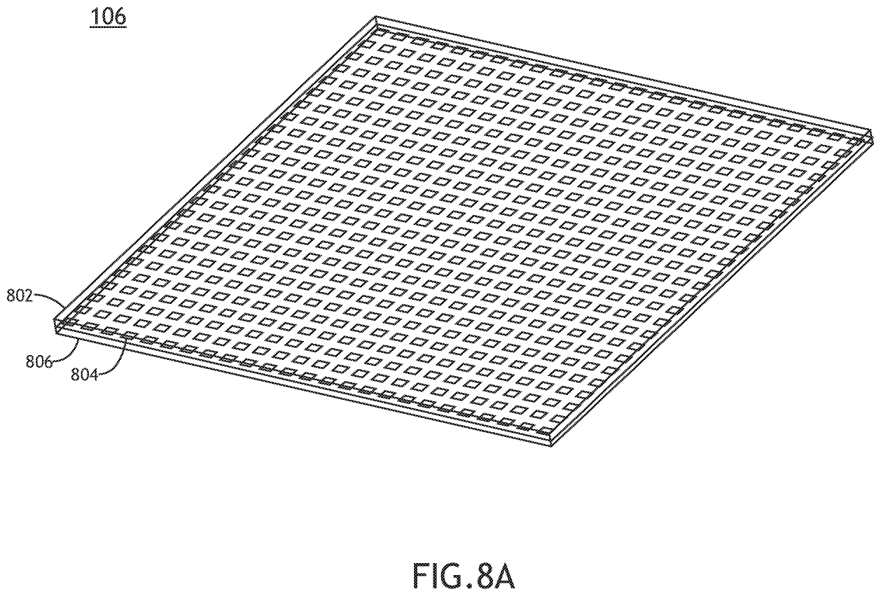

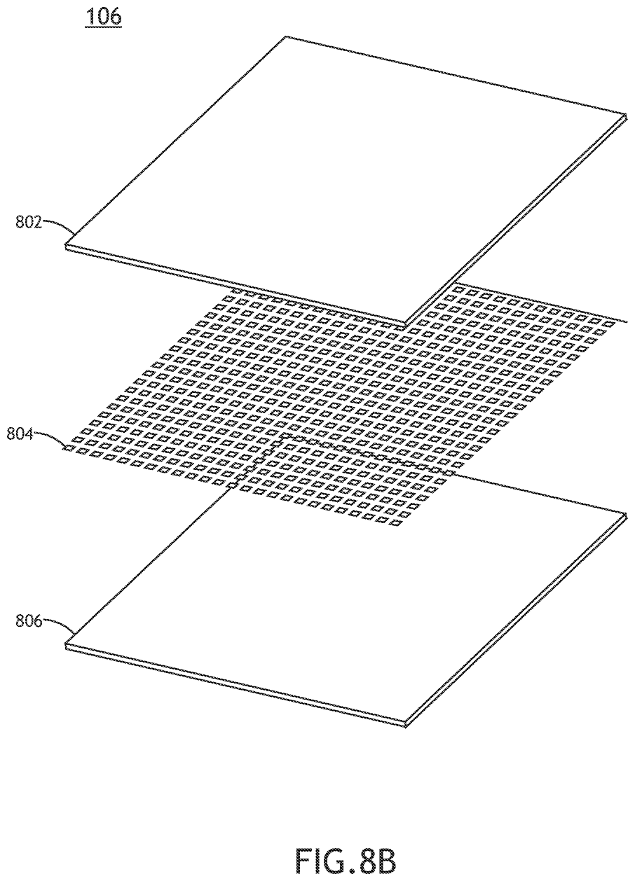

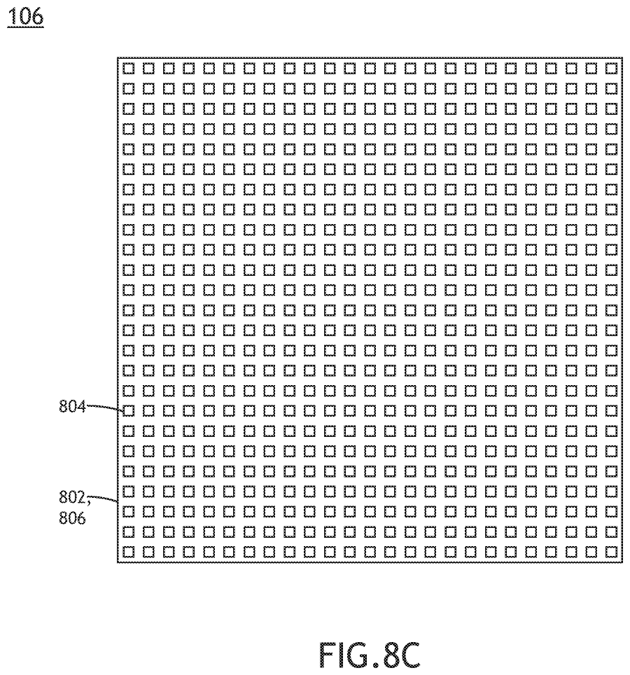

FIGS. 8A-8C illustrate an exploded view of the matching layer 106, in accordance with one or more embodiments of the present disclosure. The matching layer 106 may include one or more of an upper elastomer layer 802, a liquid-metal layer 804, and a lower elastomer layer 806.

The upper elastomer layer 802 may abut the transducer layer 104. The liquid-metal layer 804 may be disposed between the upper elastomer layer 802 and the lower elastomer layer 806. The lower elastomer layer 806 may abut the adhesive layer 108.

The liquid-metal layer 804 may include the elastomer 134 and the liquid-metal microdroplets 136 dispersed within the elastomer 134.

The liquid-metal layer 804 may be arranged in a lattice. For example, the elastomer 134 and the liquid-metal microdroplets 136 dispersed within the elastomer 134 may be arranged in a lattice. The lattice may also be referred to as a grid geometry or an array. The lattice may include a repeating pattern of the liquid-metal layer 804 along the length and/or width of the matching layer 106. The liquid-metal layer 804 may define any lattice, such as, but not limited to, an oblique lattice, a rectangular lattice, a square lattice (as depicted), or the like. The upper elastomer layer 802 may abut the lower elastomer layer 806 in the gaps between the lattice of the liquid-metal layer 804.

The upper elastomer layer 802 and/or the lower elastomer layer 806 may be made of an elastomer. The upper elastomer layer 802 and/or the lower elastomer layer 806 may be configured to conform to curved surfaces, such as a temple of a human body. The upper elastomer layer 802 and/or the lower elastomer layer 806 may be a silicone elastomer, also referred to as a silicone matrix. For example, the upper elastomer layer 802 and/or the lower elastomer layer 806 may include Polydimethylsiloxane (PDMS), polyurethane, polyvinylsiloxane, butyl rubber, fluorosilicone, styrene butadiene, or the like.

The liquid-metal layer 804 may include a selected thickness. For example, the liquid-metal layer 804 may include a thickness of 10 micrometers.

The upper elastomer layer 802 and the lower elastomer layer 806 may each include a selected thickness. The upper elastomer layer 802 may be thicker than the lower elastomer layer 806. The upper elastomer layer 802 may be stiffer than the lower elastomer layer 806. For example, the upper elastomer layer 802 may include a thickness of 160 micrometers and the lower elastomer layer 806 may include a thickness of 120 micrometers.

The upper elastomer layer 802, the liquid-metal layer 804, and/or the lower elastomer layer 806 may create a mass-spring system. The weight of the liquid-metal layer 804 may be much larger than the upper elastomer layer 802 and the lower elastomer layer 806. The liquid-metal layer 804 may be considered a mass in the mass-spring system including the weight which is much larger than the upper elastomer layer 802 and the lower elastomer layer 806.

The elastic modulus of the upper elastomer layer 802 and/or the lower elastomer layer 806 may be much larger than the liquid-metal layer 804. The upper elastomer layer 802 and/or the lower elastomer layer 806 may be considered springs by including the elastic modulus which is much larger than the liquid-metal layer 804. The upper elastomer layer 802 may be considered a stiff spring and the lower elastomer layer 806 may be considered a less stiff spring.

The matching layer 106 may provide an increased acoustic impedance matching between the wearable ultrasound patch 100 and the skin due to the arrangement of the upper elastomer layer 802, the liquid-metal layer 804, and/or the lower elastomer layer 806.

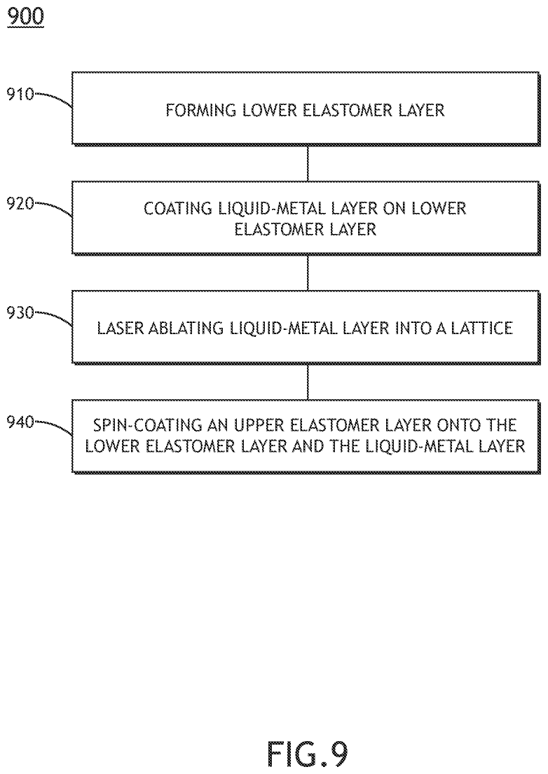

FIG. 9 illustrates a flow diagram of a method 900, in accordance with one or more embodiments of the present disclosure. The method may also be referred to as a method of manufacturing the matching layer 106. The embodiments and the enabling technologies described previously herein in the context of the matching layer 106 should be interpreted to extend to the method 900. It is further noted, however, that the method 900 is not limited to the architecture of the matching layer 106.

In a step 910, a lower elastomer layer may be formed. For example, the lower elastomer layer 806 may be formed. The lower elastomer layer 806 may be formed by an injection molding technique. The lower elastomer layer 806 may be formed of PDMS.

In a step 920, a liquid-metal layer may be coated on the lower elastomer layer. For example, the liquid-metal layer 804 may be coated on the lower elastomer layer 806. The liquid-metal layer 804 may be coated on the lower elastomer layer 806 using an airbrush to spray a liquid-metal mixture on the lower elastomer layer 806. The liquid-metal mixture may include the elastomer 134 and the liquid-metal microdroplets 136 dispersed within the elastomer 134

In a step 930, the liquid-metal layer may be laser ablated into a lattice. For example, the liquid-metal layer 804 may be laser ablated into the lattice. The liquid-metal layer 804 may be laser ablated into the lattice using a laser cutter or the like.

In a step 940, an upper elastomer layer may be spin-coated onto the lower elastomer layer and the liquid-metal layer. For example, the upper elastomer layer 802 may be spin-coated onto the lower elastomer layer 806 and the liquid-metal layer 804.

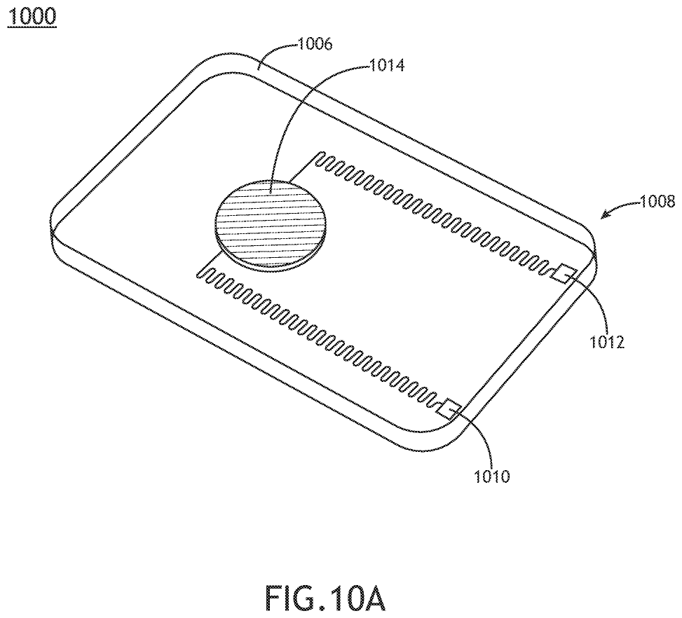

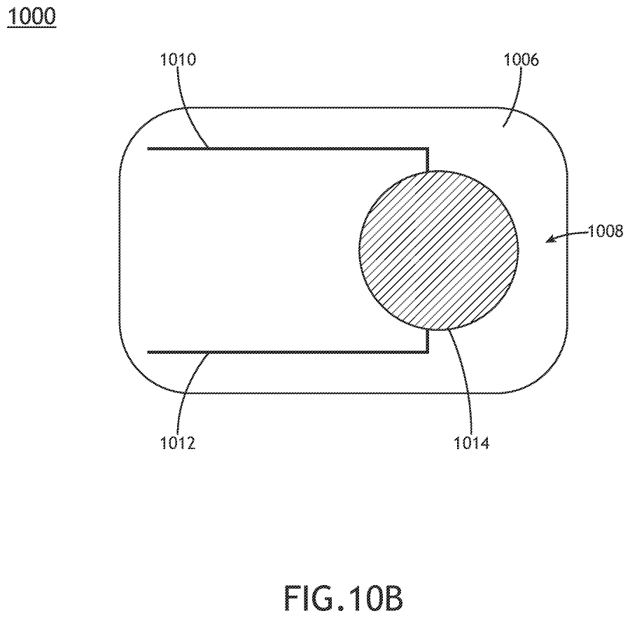

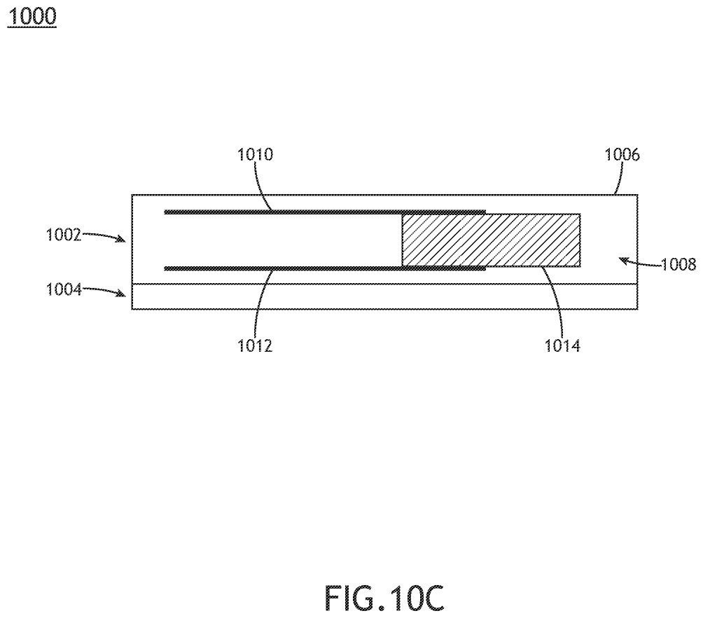

FIGS. 10A-10C illustrate a wearable ultrasound patch 1000, in accordance with one or more embodiments of the present disclosure. The wearable ultrasound patch 1000 may be an additional wearable ultrasound patch to the wearable ultrasound patch 100. The wearable ultrasound patch 1000 may include a transducer layer 1002 and an adhesive layer 1004.

The transducer layer 1002 may be disposed above the adhesive layer 1004. The transducer layer 1002 may be abutted to the adhesive layer 1004. The transducer layer 1002 may be a top-most layer of the wearable ultrasound patch 1000.

The adhesive layer 1004 may be disposed below the transducer layer 1002. The adhesive layer 1004 may be abutted to the transducer layer 1002. The adhesive layer 1004 may be adhered to the transducer layer 1002. The adhesive layer 1004 may be a bottom-most layer of the wearable ultrasound patch 1000. The adhesive layer 1004 may include a flexible skin adhesive, such as a double-sided medical adhesive.

The transducer layer 1002 and/or the adhesive layer 1004 may be conformal. For example, the transducer layer 1002 and/or the adhesive layer 1004 may be configured to conform to a planar surface and/or a curved surface. The transducer layer 1002 and/or the adhesive layer 1004 may not be rigid.

The transducer layer 1002 may include an elastomer 1006 and an electrical circuit 1008. The electrical circuit 1008 may include a top electrode 1010, a bottom electrode 1012, and a piezoelectric element 1014.

The elastomer 1006 may be a soft material. The elastomer 1006 may be configured to conform to curved surfaces, such as a neck of a human body. The elastomer 1006 may be a silicone elastomer, also referred to as a silicone matrix. For example, the elastomer 1006 may include Polydimethylsiloxane (PDMS), commercially available as Sylgard™ 184 silicon elastomer, polyurethane, polyvinylsiloxane, butyl rubber, fluorosilicone, or styrene butadiene, or the like.

The elastomer 1006 may include an acoustic impedance. For example, the elastomer 1006 may include an acoustic impedance of 1 MRayl.

The elastomer 1006 may embed at least a portion of the electrical circuit 1008. Embedding may also be referred to as encapsulating, and/or encasing. For example, the elastomer 1006 may embed the piezoelectric element 1014, a portion of the top electrode 1010, and a portion of the bottom electrode 1012. A remainder of the top electrode 1010 and/or a remainder of the bottom electrode 1012 may not be embedded by the elastomer 1006, thereby allowing for an external source to couple with the top electrode 1010 and the bottom electrode 1012.

The elastomer 1006 may be a dielectric. The elastomer 1006 may insulate electric current within the electrical circuit 1008, thereby preventing electric current flow from the transducer layer 1002.

The top electrode 1010 and the bottom electrode 1012 may be electrically conductive. The top electrode 1010 and the bottom electrode 1012 may be made of an electrically conductive material, such as, but not limited to, copper.

The piezoelectric elements 1014 may be bonded to the top electrode 1010 and/or the bottom electrode 1012. The piezoelectric elements 1014 may be bonded to the top electrode 1010 and/or the bottom electrode 1012 by a conductive epoxy. For example, the conductive epoxy may include a conductive silver epoxy, such as 8331D commercially available from MG Chemicals™.

The electrical circuit 1008 may be flexible. The top electrode 1010 and/or the bottom electrode 1012 may be flexible thereby enabling flexure of the electrical circuit 1008. For example, the top electrode 1010 and/or the bottom electrode 1012 may be serpentine-patterned electrodes. The piezoelectric element 1014 may or may not be flexible.

The electrical circuit 1008 may be configured to generate the emitted ultrasound waves 130 and/or receive the received ultrasound waves 132. The piezoelectric element 1014 may be configured to generate the emitted ultrasound waves 130 in response to receiving the input signals 131. The piezoelectric elements 1014 may generate the emitted ultrasound waves 130 by transducing the input signals 131 to the emitted ultrasound waves 130 by vibration. The piezoelectric element 1014 may be configured to generate the output signals 133 in response to receiving the received ultrasound waves 132. The piezoelectric element 1014 may generate the output signals 133 by transducing the vibration induced by the received ultrasound waves 132 to the output signals 133. Thus, the piezoelectric element 1014 may be both an ultrasound transmitter and ultrasound receiver.

The piezoelectric element 1014 may be an unfocused (flat) transducer. The piezoelectric element 1014 may include an acoustic impedance. For example, the acoustic impedance of the piezoelectric element 1014 may be 12 MRayl. The acoustic impedance of the piezoelectric element 1014 may be less than the acoustic impedance of the piezoelectric elements 118. The piezoelectric element 1014 may include a 2 MHz center frequency, 1-3 Dice & Fill Composite, PZT material 5 Amp, 150 micrometer pixels, 100 micrometer kerf width, 35% fill factor CuSn electrode poled, commercially available from Smart Material™. The 35% fill factor may be selected to provide a lowest possible acoustic impedance. Reducing the acoustic impedance of the piezoelectric element 1014 may be beneficial to provide better matching with the acoustic impedance of the elastomer 1006.

The piezoelectric element 1014 may be configured to emit the emitted ultrasound waves 130 and receive the received ultrasound waves 132. The emitted ultrasound waves 130 of the piezoelectric element 1014 may include a near-field limit. The near-field limit may be to a distance from the piezoelectric element 1014 at which the emitted ultrasound waves 130 include an axial acoustic field with a maximum intensity. The near-field limit may be within a selected near-field limit. For example, the near-field limit of the emitted ultrasound waves 130 may be between 15 and 30 millimeters. For instance, the near-field limit of the emitted ultrasound waves 130 may be 21 millimeters.

The near-field limit of the emitted ultrasound waves 130 may be based on the diameter of the piezoelectric element 1014 and a wavelength of the emitted ultrasound waves 130 through an acoustic media. For example, the near-field limit of the emitted ultrasound waves 130 may defined by the following formula:

$${Z}_{nf} = \frac{{D}^{2}}{4 \cdot λ}$$

Where Z_{nf} represents the distance at which the axial acoustic field of an unfocused (flat) piezoelectric transducer has maximum intensity, D is the diameter of the piezoelectric elements 118, and λ is the wavelength of the emitted ultrasound waves 130 at the resonant frequency of the piezoelectric element 1014. The diameter of the piezoelectric element 1014 and the center frequency of the emitted ultrasound waves 130 may be selected so that the near-field limit is within the selected near-field limit. For example, the piezoelectric element 1014 with the diameter of 8 millimeters and a center frequency of the emitted ultrasound waves 130 at 2 MHz, emitting into human tissue with a speed of sound (c) of 1540 meters per second, Z_{nf} may be 21 millimeters.

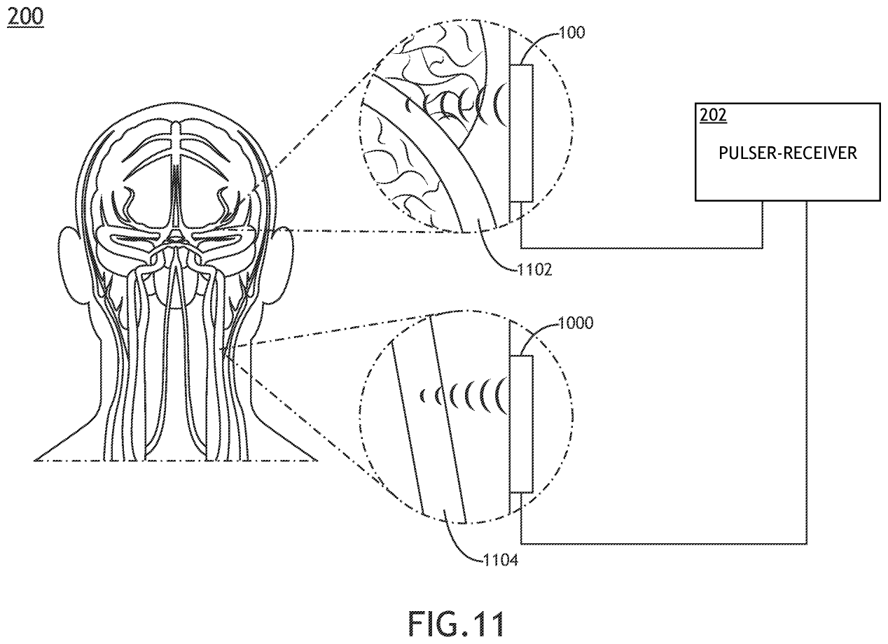

FIG. 11 illustrates the system 200, in accordance with one or more embodiments of the present disclosure. The system 200 may include the wearable ultrasound patch 100, the pulser-receiver 202, the patient 204, and/or the wearable ultrasound patch 1000.

The patient 204 may include one or more arteries. For example, the patient 204 may include a middle cerebral artery 1102 (MCA) and/or an internal carotid artery 1104 (ICA).

The wearable ultrasound patch 100 may be placed over the temple of the patient 204. The wearable ultrasound patch 100 may measure a mean velocity of blood flow (\tilde{v}_{MCA}) in the middle cerebral artery 1102.

The wearable ultrasound patch 1000 may be placed over the neck of the patient 204. The wearable ultrasound patch 1000 may measure a mean velocity of blood flow (\tilde{v}_{ICA}) in the internal carotid artery 1104.

The pulser-receiver 202 may cause the wearable ultrasound patch 100 and the wearable ultrasound patch 1000 to obtain measurements of the mean velocity of blood flow (\tilde{v}_{MCA}) in the middle cerebral artery 1102 and the mean velocity of blood flow (\tilde{v}_{ICA}) in the internal carotid artery 1104, respectively, for calculating the Lindegaard ratio.

The patient 204 may experience a vasospasm. The vasospasm may refer to a constriction of the middle cerebral artery 1102. For example, the patient 204 may be at risk for the vasospasm after an aneurysmal subarachnoid hemorrhage (aSAH). A method for detecting the vasospasm may be to compare the mean velocity of blood flow (\tilde{v}_{MCA}) in the middle cerebral artery 1102 with the mean velocity of blood flow (\tilde{v}_{ICA}) in the internal carotid artery 1104. A Lindegaard Ratio may be a mean velocity of blood flow (\tilde{v}_{MCA}) in the middle cerebral artery 1102 over the mean velocity of blood flow (\tilde{v}_{ICA}) in the internal carotid artery 1104. A Lindegaard ratio greater than 3 may indicate a vasospasm.

The pulser-receiver 202 may receive the measurements of the mean velocity of blood flow (\tilde{v}_{MCA}) in the middle cerebral artery 1102 and the mean velocity of blood flow (\tilde{v}_{ICA}) in the internal carotid artery 1104 and calculate the Lindegaard ratio. Thus, the system 200 may be used for detection of vasospasm secondary to aSAH. The pulser-receiver 202 may generate one or more alerts in response to the Lindegaard ratio being greater than 3.

The measurements of the mean velocity of blood flow (\tilde{v}_{MCA}) in the middle cerebral artery 1102 and the mean velocity of blood flow (\tilde{v}_{ICA}) in the internal carotid artery 1104 may be measured at any interval. The interval may be selected to be frequent enough to detect the vasospasm in time for intervention, yet far enough apart and with low enough duty cycle to minimize exposure to acoustic radiation and risk of excessive tissue heating. The interval may include, but is not limited to, every 15 minutes.

The pulser-receiver 202 may excite the wearable ultrasound patch 100 and/or the wearable ultrasound patch 1000 with volts, for a select duration, with a select pulse-repetition frequency, a select energy setting, a select damping setting, a select gain, one or more filters, and the like. For example, the pulser-receiver 202 may excite the wearable ultrasound patch 100 and/or the wearable ultrasound patch 1000 with 500 volts, for a 1 microsecond duration, with a 500 Hz pulse-reputation frequency, with an energy setting of 4, with a damping setting of 3, with a gain of 55 dB, with a high pass filter at 1 MHZ, and/or with a low pass filter at a full bandwidth.

The pulser-receiver 202 may calculate the mean velocity of blood flow (\tilde{v}_{MCA}) in the middle cerebral artery 1102 and the mean velocity of blood flow (\tilde{v}_{ICA}) in the internal carotid artery 1104 using the output signals 133 from the wearable ultrasound patch 100 and the output signals 133 from the wearable ultrasound patch 1000, respectively. The pulser-receiver 202 may calculate the mean velocity of blood flow (\tilde{v}_{MCA}) in the middle cerebral artery 1102 and the mean velocity of blood flow (\tilde{v}_{ICA}) in the internal carotid artery 1104 by performing one or more steps.

For example, N-number of consecutive A-lines may be extracted from the output signals 133, where N is an integer. Each of the N-number of consecutive A-lines may be multiplied by a complex exponential of the center frequency of the emitted ultrasound waves 130 to shift the fast-Fourier transform of the A-line so that one of the spectral peaks is centered at 0 Hz. A finite impulse response (FIR) lowpass filter (order=8) may be applied to each of the N-number of consecutive A-lines to remove the peak that is not at 0 Hz. The passband frequencies (fₚₐₛₛ) for the FIR lowpass filter may be the center frequency (f₀) divided by twice the sampling frequency (fₛ). The stopband frequencies (fₛₜₒₚ) for the FIR lowpass filter may be the center frequency (f₀) divided by the sampling frequency (fₛ).

After lowpass filtering, a section of each of the N-number of consecutive A-lines may be summed to generate a complex-valued number. The complex-valued number may include N-number of the complex numbers. A fast-Fourier transform of the complex-valued number may generate a spectrum representing the motion of the blood in the middle cerebral artery 1102 and/or the internal carotid artery 1104.

A FIR high pass filter, such as a wall filter, may be applied to remove low-frequency vibration of the middle cerebral artery 1102 and/or the internal carotid artery 1104. The FIR high pass filter may include an order=15, a maximum stopband frequency 5 Hz, and a cutoff frequency of 10 Hz.

A N-point Hanning window may then be applied. A frequency spectrum F(f) may be estimated by taking the N-point fast-Fourier transform of the Hanning-windowed data. A logarithmic power spectrum P (f) may be calculated from the frequency spectrum F(f) using the following equation:

$$P ( f ) = {log}_{10} ( F ( f ) \cdot {F}^{*} ( f ) )$$

Where the * may denote the complex conjugate. The Doppler equation may convert the frequency to velocity using the following equation:

$${f}_{d} = \frac{2 \cdot v \cdot cos ( θ )}{c} {f}_{0}$$

Where v is the velocity of the blood, c is the speed of sound in the medium through which the sound is travelling (c may be 1840 m/s for water at 20 degrees Celsius), Θ is the angle between the direction of sound propagation and the direction of flow, and f₀ is the center frequency. Thus, the mean velocity of blood flow (\tilde{v}_{MCA}) in the middle cerebral artery 1102 and the mean velocity of blood flow (\tilde{v}_{ICA}) in the internal carotid artery 1104 may be measured.

It is further contemplated that each of the embodiments of the method described above may include any other step(s) of any other method(s) described herein. In addition, each of the embodiments of the method described above may be performed by any of the systems described herein.

The wearable ultrasound patch 100 may also be configured to measure other vital signs such as body temperature, respiration rate, and the like. For example, the wearable ultrasound patch 100 may include sensors, such as, but not limited to, temperature sensors, respiration sensors, and the like.

One skilled in the art will recognize that the herein described components operations, devices, objects, and the discussion accompanying them are used as examples for the sake of conceptual clarity and that various configuration modifications are contemplated. Consequently, as used herein, the specific exemplars set forth and the accompanying discussion are intended to be representative of their more general classes. In general, use of any specific exemplar is intended to be representative of its class, and the non-inclusion of specific components, operations, devices, and objects should not be taken as limiting.

As used herein, directional terms such as “top,” “bottom,” “over,” “under,” “upper,” “upward,” “lower,” “down,” and “downward” are intended to provide relative positions for purposes of description, and are not intended to designate an absolute frame of reference. Various modifications to the described embodiments will be apparent to those with skill in the art, and the general principles defined herein may be applied to other embodiments

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “connected,” or “coupled,” to each other to achieve the desired functionality, and any two components capable of being so associated can also be viewed as being “couplable,” to each other to achieve the desired functionality. Specific examples of couplable include but are not limited to physically mixable and/or physically interacting components and/or wirelessly interactable and/or wirelessly interacting components and/or logically interacting and/or logically interactable components.

Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” and the like). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, and the like” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, and the like). In those instances where a convention analogous to “at least one of A, B, or C, and the like” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, and the like). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is believed that the present disclosure and many of its attendant advantages will be understood by the foregoing description, and it will be apparent that various changes may be made in the form, construction and arrangement of the components without departing from the disclosed subject matter or without sacrificing all of its material advantages. The form described is merely explanatory, and it is the intention of the following claims to encompass and include such changes. Furthermore, it is to be understood that the invention is defined by the appended claims.

What is claimed:

1. A wearable ultrasound patch comprising:
a transducer layer comprising an electrical circuit and a first elastomer, the electrical circuit comprising a ground plane, a plurality of feed lines, and a plurality of piezoelectric elements, wherein the ground plane comprises a plurality of ground electrodes and a plurality of ground traces, wherein the plurality of feed lines comprise a plurality of feed electrodes, a plurality of feed traces, and an interface, wherein the plurality of piezoelectric elements couple the plurality of feed electrodes and the plurality of ground electrodes, wherein the plurality of piezoelectric elements are configured to generate a plurality of emitted ultrasound waves in response to receiving a plurality of input signals and generate a plurality of output signals in response to receiving a plurality of received ultrasound waves; and
a matching layer comprising a second elastomer and a plurality of liquid-metal microdroplets, wherein the plurality of liquid-metal microdroplets are dispersed within the second elastomer.

2. The wearable ultrasound patch of claim 1, comprising a backing layer, wherein the transducer layer is disposed between the backing layer and the matching layer.

3. The wearable ultrasound patch of claim 2, wherein the backing layer is abutted to the transducer layer.

4. The wearable ultrasound patch of claim 1, comprising an adhesive layer, wherein the matching layer is disposed between the transducer layer and the adhesive layer.

5. The wearable ultrasound patch of claim 4, wherein at least one of:
the matching layer is abutted to the transducer layer; or
the adhesive layer is abutted to the matching layer.

6. The wearable ultrasound patch of claim 1, wherein the first elastomer embeds the ground plane, the plurality of piezoelectric elements, the plurality of feed electrodes, and the plurality of feed traces.

7. The wearable ultrasound patch of claim 1, wherein the transducer layer and the matching layer are configured to conform to a planar surface and a curved surface.

8. The wearable ultrasound patch of claim 1, wherein the matching layer comprises an elastic modulus of between 10 kPa and 100 MPa.

9. The wearable ultrasound patch of claim 8, wherein the matching layer comprises the elastic modulus of between 10 kPa and 1 MPa.

10. The wearable ultrasound patch of claim 1, wherein the plurality of liquid-metal microdroplets comprise a diameter between 10 nanometers and 500 micrometers.

11. The wearable ultrasound patch of claim 10, wherein the plurality of liquid-metal microdroplets comprise the diameter between 100 nanometers and 100 micrometers.

12. The wearable ultrasound patch of claim 1, wherein the matching layer comprises an acoustic impedance between 1 and 7.2 MRayls.

13. The wearable ultrasound patch of claim 12, wherein the matching layer comprises the acoustic impedance between 4.8 and 7.2 MRayls.

14. The wearable ultrasound patch of claim 13, wherein the matching layer comprises the acoustic impedance between 5.6 and 5.8 MRayls.

15. The wearable ultrasound patch of claim 1, wherein the plurality of liquid-metal microdroplets comprise a volume loading between 10% and 80%.

16. The wearable ultrasound patch of claim 15, wherein the plurality of liquid-metal microdroplets comprise the volume loading between 50% and 70%.

17. The wearable ultrasound patch of claim 1, wherein at least one of the first elastomer and the second elastomer comprises at least one of Polydimethylsiloxane, polyurethane, polyvinylsiloxane, butyl rubber, fluorosilicone, or styrene butadiene.

18. The wearable ultrasound patch of claim 1, wherein the plurality of liquid-metal microdroplets comprise a eutectic gallium-based liquid metal.

19. The wearable ultrasound patch of claim 18, wherein the eutectic gallium-based liquid metal comprises one of eutectic gallium-indium (EGaIn) or a eutectic alloy of gallium, indium, and tin.

20. The wearable ultrasound patch of claim 18, wherein the plurality of liquid-metal microdroplets comprise a core and a liquid-metal shell, wherein the liquid-metal shell comprises an alloy of a first metal and the eutectic gallium-based liquid metal.

21. The wearable ultrasound patch of claim 20, wherein the core comprises at least one of tungsten, tungsten carbide, aluminum, copper, iron, nickel, or silver.

22. The wearable ultrasound patch of claim 20, wherein the first metal comprises at least one of silver, copper, nickel, gold, or tin.

23. The wearable ultrasound patch of claim 20, wherein the plurality of liquid-metal microdroplets comprise an adhesive shell, wherein the adhesive shell is disposed between the core and the liquid-metal shell.

24. The wearable ultrasound patch of claim 23, wherein the adhesive shell comprises polydopamine (PDA).

25. The wearable ultrasound patch of claim 24, wherein the eutectic gallium-based liquid metal comprises eutectic gallium-indium (EGaIn), wherein the core is a tungsten (W) core, wherein the first metal is silver (Ag), wherein the plurality of liquid-metal microdroplets are W@PDA-(Ag+EGaIn) microdroplets.

26. The wearable ultrasound patch of claim 1, wherein the wearable ultrasound patch is configured to perform acoustic beamforming of the plurality of piezoelectric elements by receiving the plurality of input signals at separate phases.

27. The wearable ultrasound patch of claim 1, wherein the plurality of piezoelectric elements are arranged in an array, wherein the array is a planar array.

28. The wearable ultrasound patch of claim 27, wherein the array is a five-row array with one of the plurality of piezoelectric elements in a first row, five of the plurality of piezoelectric elements in a second row, six of the plurality of piezoelectric elements in a third row, five of the plurality of piezoelectric elements in a fourth row, and two of the plurality of piezoelectric elements in a fifth row.

29. The wearable ultrasound patch of claim 1, wherein the matching layer comprises an upper elastomer layer, a liquid-metal layer, and a lower elastomer layer, wherein the liquid-metal layer comprises the second elastomer and the plurality of liquid-metal microdroplets, wherein the liquid-metal layer is disposed between the upper elastomer layer and the lower elastomer layer.

30. The wearable ultrasound patch of claim 29, wherein the liquid-metal layer is arranged in a lattice.

31. The wearable ultrasound patch of claim 29, wherein the upper elastomer layer abuts the transducer layer, wherein upper elastomer layer is thicker than the lower elastomer layer.

32. A system comprising:
a wearable ultrasound patch comprising:
a transducer layer comprising an electrical circuit and a first elastomer, the electrical circuit comprising a ground plane, a plurality of feed lines, and a plurality of piezoelectric elements, wherein the ground plane comprises a plurality of ground electrodes and a plurality of ground traces, wherein the plurality of feed lines comprise a plurality of feed electrodes, a plurality of feed traces, and an interface, wherein the plurality of piezoelectric elements couple the plurality of feed electrodes and the plurality of ground electrodes, wherein the plurality of piezoelectric elements are configured to generate a plurality of emitted ultrasound waves in response to receiving a plurality of input signals and generate a plurality of output signals in response to receiving a plurality of received ultrasound waves; and
a matching layer comprising a second elastomer and a plurality of liquid-metal microdroplets, wherein the plurality of liquid-metal microdroplets are dispersed within the second elastomer; and
a pulser-receiver, wherein the interface is connected to the pulser-receiver, wherein the pulser-receiver is configured to transmit the plurality of input signals to the interface and receive the plurality of output signals from the interface.

33. The system of claim 32, wherein the pulser-receiver is configured to cause the wearable ultrasound patch to obtain one or more measurements of a mean velocity (\tilde{v}_{MCA}) of blood flow in a middle cerebral artery.

34. The system of claim 33, comprising an additional wearable ultrasound patch; wherein the pulser-receiver is configured to cause the additional wearable ultrasound patch to obtain one or more measurements of a mean velocity (\tilde{v}_{ICA}) of blood flow in an internal carotid artery.

35. The system of claim 34, wherein the additional wearable ultrasound patch comprises an additional transducer layer, wherein the additional transducer layer comprises an additional elastomer and an additional electrical circuit, wherein the additional electrical circuit comprises a top electrode, a bottom electrode, and a piezoelectric element.

36. A method comprising:
spin-processing a matching layer, the matching layer comprising an elastomer and a plurality of liquid-metal microdroplets, wherein the plurality of liquid-metal microdroplets are dispersed within the elastomer;
laser patterning a ground plane and a plurality of feed lines from one or more copper clad sheets, wherein the ground plane comprises a plurality of ground electrodes and a plurality of ground traces, wherein the plurality of feed lines comprise a plurality of feed electrodes, a plurality of feed traces, and an interface;
transferring the ground plane to the matching layer and the plurality of feed lines to a backing layer;
forming a conductive epoxy on the plurality of ground electrodes and on the plurality of feed electrodes;
placing a plurality of piezoelectric transducers between the plurality of ground electrodes and the plurality of feed electrodes;
curing the conductive epoxy to bond the plurality of piezoelectric transducers between the plurality of ground electrodes and the plurality of feed electrodes thereby forming an electrical circuit of a transducer layer; and
forming an elastomer of the transducer layer.
